SEQUENCE LISTING (35 sequences)

SEQ ID NO: 1 - DNA, 4099 bp, Artificial Sequence
  pIDV
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctgtctgacgctcagtggaacgaaaactcacgttaagggattttggtcatgagattatcaaaaaggatcttcacctagatccttttaaattaaaaatgaagttttagcacgtgctattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaaggcatgcctgctactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggccgcgcgccagccggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcatcgatgcatggtacc

SEQ ID NO: 2 - DNA, 366 bp, Artificial Sequence
  CMV enhancer
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 3 - DNA, 1290 bp, Artificial Sequence
  Chicken beta actin promoter and chimeric intron
tcgaggtgagccccacgttctgcttcactctccccatctcccccccctccccacccccaattttgtatttatttattttttaattattttgtgcagcgatgggggcgggggggggggggccgcgcgccagccggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 4 - DNA, 56 bp, Artificial Sequence
  beta-globin poly(A) signal
aataaaggaaatttattttcattgcaatagtgtgttggaattttttgtgtctctca

SEQ ID NO: 5 - DNA, 326 bp, Artificial Sequence
  3′ flanking region of rabbit beta-globin
ctcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 6 - DNA, 589 bp, Artificial Sequence
  ORI
tttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 7 - DNA, 76 bp, Artificial Sequence
  AmpR promoter
tttatcagggttattgtctcatgagcggatacatatttgaatgtatttagaaaaataaacaaataggggttccgcg

SEQ ID NO: 8 - DNA, 795 bp, Artificial Sequence
  NeoR/KanR
tcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcat

SEQ ID NO: 9 - DNA, 50 bp, Artificial Sequence
  NeoR/KanR promoter
ttgcagggcttcccaaccttaccagagggcgccccagctggcaattccgg

SEQ ID NO: 10 - DNA, 2999 bp, Artificial Sequence
  pVAX1
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 11 - DNA, 48 bp, Artificial Sequence
  Neo/Kan forward primer
gtaattgattactattaataactagtagcaggcatgccttctactggg

SEQ ID NO: 12 - DNA, 48 bp, Artificial Sequence
  ORI reverse primer
atccctcgacctgcagcccaagcttgttgctggcgtttttccataggc

SEQ ID NO: 13 - DNA, 5067 bp, Artificial Sequence
  CCHF glycoprotein precursor (CCHF GP-Turkey kk06)
atgcctaccaatatcatgcatacacccctcgtgtgctttatactttacctccaattgttgtgcttgggtggggcccacggacaattgaacgccaccgaacacaatgggacgaacaataccactgctcccggcgctagtcaatctcctaaacctcccatgagcaccacgcctccacatgcgccagaatcatcaacaatcaagcccacgacacctatctccgaggcggaggggtcaggagagactacgtcacccccgaataccacgcagggcctgtcttctccggaaaccacttccgaaaggccagcaactacgagcattagtactagcagtaccgattccacgaacccaacgacacaaatgacggacaatactcctacgccaacagttagtacatcccccagctccagtccttcaacccctagtactccgcagggcatccaccatccagcgagatccctcctgtctgtcagtagcccaaagactgtcacgacaccaacgccgacctctcccggagagatgtcttctgagacttcttcacagcatagcgcgatgtcaagaatcccgactccccacacagcgacgcgcgtttcaacagaaatcacaaaccaccggactccgcgacaatctgagtcatctgctcaacaaactactccttctcctatgacgtctcctgcccagtccattttgcttatgagcgcagctccaactgcggtccaggacatccacccttcccctactaataggtccaagcgaaaccttgagacagaaattattctcacactctcccaggggttgaaaaaatattacggcaaaatcctgaaactgttgcatctgaccctcgagcaagacactgaaggcctcctcgagtggtgtaagggtaatttgggcagcaattgtgatgatgatttcttccaaaaaaggatcgaggaattctttatgaccggcgagtgctattttaatgaggtccttcagttcaagacactgagtaccctctcacctaccgaaccttcacacgccaggcttccgacagccgaaccttttaaaagttacttcgcaaagggctttttgagtatcgactccggatacttcagtgctaagtgctaccctcggtcaagcgcatccgggctccagttgattaatgttacacagcatcccgcacggatcgcagagacaccaggtccgaaaactacttccttgaagacaatcaactgcattaacctgcgagcttccgtcttcaaagagcatagagaagtcgaaataaatgtacttctcccccagatcgcagttaacttgtcaaattgtcacgtcgtaatcaacagccatgtttgtgactattcccttgataccgatggtcccgttcgactccctaggatataccacgaaggaacattcataccgggcacttataagatcgtaatcgataagaagaataaacttaatgaccggtgtacgctggtcaccaattgcgtgatcaaaggtagagaggtgagaaaaggacaaagtgtgttgcgacaatataagacggaaatcaaaattggcaaggcttccacggggttcagaaaactgctcagtgaggagccgggcgacgactgcatctccaggacgcaacttttgagaacagagacagccgagattcatgatgataattatggaggacctggagacaaaataacgatatgtaacggctccactatagtcgaccaaagattgggaagcgagcttggttgctacacgattaacagagtgaagtcttttaaactttgtaagaacagtgccacggggaagacatgtgaagttgactccaccccagtgaaatgccgacaaggattttgtctgaagattacacaagaaggtcgaggccacgtcaaactttcaaggggaagtgaagtcgttttggacgcttgcgattcatcctgcgaagtaatgattccaaaaggtactggagacattttggtggattgttcaggcgggcaacaacactttctgaaagacaacctgatagacctcggatgccctcatatccccctgcttggtaggatggcaatctatatctgtcggatgtcaaatcatcctcgaactacgatggcattcctcttttggttctcttttggttacgtcattacatgcattttctgtaaggcactcttttattcactcattatcatcgggacacttggaaaaaaaatcaaacaataccgggaacttaagccccaaacctgcaccatttgcgaaactgcgccagtaaacgcaatcgatgccgaaatgcatgatctcaattgttcatacaacatatgtccctactgtgcgagcaggcttactagtgatggtttggcacgacacgtaactcagtgtcctaagagaaaagaaaaagtcgaagaaacagaattgtacctcaacctcgaacgaataccttggatcgtaaggaagctcctgcaggtctcagagagtaccggggtcgctcttaaaagaagctcatggctgatcgtacttttggtcctcctcactgtatcactgtcccctgtccaaagcgccccagtcggacatggcaaaactattgagatatatcaaacgcgagaagggtttgcatctatttgtctttttatgctcggcagcatcctcttcatagtgtcttgcttggtcaagggactggtagacagcgtgtctgaatctttctttcctgggctctcagtctgtaagacttgttccattggcagtgtcaacggtttcgagattgaatcccacaagtgttattgcagtttgttttgctgcccatactgccgccattgtagcgcggatcgagaaattcatcaactgcaccttagcatttgcaaaaagaggaaaacgggctcaaacgtaatgctcgcggtctgcaaacgaatgtgtttcagagcgacaattgaggcaagtcggcgagcactgctgattaggagtattattaatactacgtttgtgatctgtatcctcacgctgactatatgcgttgtcagtacatccgccgtcgagatggaaaatcttcctgcagggacgtgggagcgagaggaagatcttactaatttttgccatcaagaatgtcaagtgacagaaactgaatgcttgtgcccctatgaagcccttgtgttgaggaagccactctttcttgattctatagttaagggaatgaaaaatttgctcaacagtacgagtctggaaacctctttgtcaatcgaggccccatggggtgccatcaacgtgcagagtacgtttaagccaaccgtttcaaccgctaacatcgctcttagctggtcaagcgtcgttcatagaggaaacaagatacttgtcactggacggagtgaaagcatcatgaagcttgaggaaaggactggggtgagttgggatcttggggtcgaggacgcatctgaaagtaaactgttgacggtctctatcatggatctctcacagatgtacagcccggtttttgagtacctcagcggggatcgacaagttgaggagtggcccaaggctacctgcaccggagattgcccggagcgatgcgggtgcactagctcaacatgtctccataaggagtggtcccatagccgaaattggaggtgtaaccccacttggtgctggggtgtcggcacaggatgtacttgttgcggagtcgatgtaaaagacctgttcacggaccatatgtttgtcaaatggaaggtggagtacattaaaactgaagccattgtgtgcgttgcgcttacgtctcaagaacggcaatgttcactgatcgaagcagggactcggttcaacttggggcctgtcacaataactttgtcagagccgagaaacatacaacagaagcttccacccgaaatcataaccttgcatccgaaaatagaggaagggttcttcgatcttatgcacgtccaaaaagtgcttagtgcctctacggtctgcaaactccaaagctgcactcacggtatcccgggggatctgcaagtttaccacataggcaaccttttgaaaggagatagggtcaacggccacctgatacataagattgaaagccatttcaacacaagttggatgagttgggatggctgcgatttggattattactgcaatatgggggactggcccagctgtacatatactggagttacacagcataatcatgcggcatttgttaacctccttaacatcgagacagactacacaaagacctttcacttccactccaagagagtgactgcgcacggagacacgccccaacttgaccttaaagctagaccgacctacggagcgggcggaatcacagtcctggttgaagtagctgatatggaattgcatacgaaaaaggttgagattagtgggctcaagttcgcgtcactcgcttgcacgggttgctatgcgtgttccagcggcattagctgcaaggtcagaatccatgttgacgagccggatgagttgacagtacatgtcaaatccagtgacccagacgttgtcgctgcaagtacatccctgatggcgaggaagctggaattcggcacggactccacgttcaaagcgttttcagcgatgccgaagacgtctttgtgtttctatattgtagagcgagaatattgtaaaagctgcagtgaagatgacactcagaaatgcgttgatactcgcttggaacagcctcagtcaattctcatagaacataagggaacaattatcggaaaacagaacgatacttgtaccgccaaagcatcctgttggctggaaagtgtgaagtcttttttctatggcctgaaaaacatgcttgggagtgtcttcgggaatttgttcatcggcatactgcttttcttggccccctttgtcctcctcgtcctcttcttcatgttcggatggaagatactgttttgcttcaaatgctgcagacgcactagggggctgttcaagtatcgacatctgaaggacgacgaagagaccgggtaccgaaggattatcgagagactcaattctaaaaagggcaaaaatcggttgcttgacggggaccgcttggcagacaggaagatcgcagagttgttctccacgaaaacacatatcggataa

SEQ ID NO: 14 - DNA, 2163 bp, Artificial Sequence
  Ubiquitin-CCHF glycoprotein GC
atgcagatcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggttttcttgattctatagttaagggaatgaaaaatttgctcaacagtacgagtctggaaacctctttgtcaatcgaggccccatggggtgccatcaacgtgcagagtacgtttaagccaaccgtttcaaccgctaacatcgctcttagctggtcaagcgtcgttcatagaggaaacaagatacttgtcactggacggagtgaaagcatcatgaagcttgaggaaaggactggggtgagttgggatcttggggtcgaggacgcatctgaaagtaaactgttgacggtctctatcatggatctctcacagatgtacagcccggtttttgagtacctcagcggggatcgacaagttgaggagtggcccaaggctacctgcaccggagattgcccggagcgatgcgggtgcactagctcaacatgtctccataaggagtggtcccatagccgaaattggaggtgtaaccccacttggtgctggggtgtcggcacaggatgtacttgttgcggagtcgatgtaaaagacctgttcacggaccatatgtttgtcaaatggaaggtggagtacattaaaactgaagccattgtgtgcgttgcgcttacgtctcaagaacggcaatgttcactgatcgaagcagggactcggttcaacttggggcctgtcacaataactttgtcagagccgagaaacatacaacagaagcttccacccgaaatcataaccttgcatccgaaaatagaggaagggttcttcgatcttatgcacgtccaaaaagtgcttagtgcctctacggtctgcaaactccaaagctgcactcacggtatcccgggggatctgcaagtttaccacataggcaaccttttgaaaggagatagggtcaacggccacctgatacataagattgaaagccatttcaacacaagttggatgagttgggatggctgcgatttggattattactgcaatatgggggactggcccagctgtacatatactggagttacacagcataatcatgcggcatttgttaacctccttaacatcgagacagactacacaaagacctttcacttccactccaagagagtgactgcgcacggagacacgccccaacttgaccttaaagctagaccgacctacggagcgggcggaatcacagtcctggttgaagtagctgatatggaattgcatacgaaaaaggttgagattagtgggctcaagttcgcgtcactcgcttgcacgggttgctatgcgtgttccagcggcattagctgcaaggtcagaatccatgttgacgagccggatgagttgacagtacatgtcaaatccagtgacccagacgttgtcgctgcaagtacatccctgatggcgaggaagctggaattcggcacggactccacgttcaaagcgttttcagcgatgccgaagacgtctttgtgtttctatattgtagagcgagaatattgtaaaagctgcagtgaagatgacactcagaaatgcgttgatactcgcttggaacagcctcagtcaattctcatagaacataagggaacaattatcggaaaacagaacgatacttgtaccgccaaagcatcctgttggctggaaagtgtgaagtcttttttctatggcctgaaaaacatgcttgggagtgtcttcgggaatttgttcatcggcatactgcttttcttggccccctttgtcctcctcgtcctcttcttcatgttcggatggaagatactgttttgcttcaaatgctgcagacgcactagggggctgttcaagtatcgacatctgaaggacgacgaagagaccgggtaccgaaggattatcgagagactcaattctaaaaagggcaaaaatcggttgcttgacggggaccgcttggcagacaggaagatcgcagagttgttctccacgaaaacacatatcggataa

SEQ ID NO: 15 - DNA, 1092 bp, Artificial Sequence
  Ubiquitin-CCHF Glycoprotein Gn
atgcagatcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggtagtgaggagccgggcgacgactgcatctccaggacgcaacttttgagaacagagacagccgagattcatgatgataattatggaggacctggagacaaaataacgatatgtaacggctccactatagtcgaccaaagattgggaagcgagcttggttgctacacgattaacagagtgaagtcttttaaactttgtaagaacagtgccacggggaagacatgtgaagttgactccaccccagtgaaatgccgacaaggattttgtctgaagattacacaagaaggtcgaggccacgtcaaactttcaaggggaagtgaagtcgttttggacgcttgcgattcatcctgcgaagtaatgattccaaaaggtactggagacattttggtggattgttcaggcgggcaacaacactttctgaaagacaacctgatagacctcggatgccctcatatccccctgcttggtaggatggcaatctatatctgtcggatgtcaaatcatcctcgaactacgatggcattcctcttttggttctcttttggttacgtcattacatgcattttctgtaaggcactcttttattcactcattatcatcgggacacttggaaaaaaaatcaaacaataccgggaacttaagccccaaacctgcaccatttgcgaaactgcgccagtaaacgcaatcgatgccgaaatgcatgatctcaattgttcatacaacatatgtccctactgtgcgagcaggcttactagtgatggtttggcacgacacgtaactcagtgtcctaagagaaaagaaaaagtcgaagaaacagaattgtacctcaacctcgaacgaataccttggatcgtaaggaagctcctg

SEQ ID NO: 16 - DNA, 1449 bp, Artificial Sequence
  CCHF Nucleoprotein (NP)
atggaaaacaagatcgaggtgaataacaaagatgagatgaacaggtggtttgaagagttcaaaaaaggaaatggacttgtggacaccttcacaaactcctattccttttgcgagagtgttcccaatttggacaggtttgtgtttcagatggccagtgccaccgatgatgcacagaaggactccatctacgcatctgctctggtggaggcaacaaagttttgtgcacctatatatgagtgcgcatgggttagctccactggcattgtaaaaaagggacttgaatggttcgagaaaaatgcaggaaccattaagtcctgggatgaaagttatactgagctaaaggtcgacgtcccgaaaatagagcagcttaccggttaccaacaagctgccttgaagtggagaaaagacataggtttccgtgtcaatgccaacacagcagctctgagcaacaaagtcctcgcagaatacaaagtccctggtgagattgtgatgtctgtcaaagagatgctgtcagacatgattaggagaaggaacctgattctaaacaggggtggtgatgagaacccacgtggcccagtgagccatgagcatgtagactggtgcagggagtttgtcaaaggcaaatacatcatggccttcaacccaccatggggggacatcaacaagtcaggccgttcaggaatagcacttgttgcaacaggccttgctaagcttgcagagactgaaggaaagggaatatttgatgaagccaaaaagactgtggaggccctcaacgggtatctggacaagcataaggacgaagttgatagagcaagcgccgacagcatgataacaaaccttcttaagcatattgccaaggcacaggagctctataaaaattcatctgcacttcgtgcacaaagcgcacagattgacactgctttcagctcatactattggctttacaaggctggcgtgactcctgaaaccttcccgacggtgtcacagttcctctttgagctagggaaacagccaagaggtaccaagaaaatgaagaaggctcttctgagcaccccaatgaagtgggggaagaagctttatgagctctttgccgatgattctttccagcagaacaggatttacatgcatcctgccgtgcttacagctggtagaatcagtgaaatgggagtctgctttgggacaatccctgtggccaatcctgatgatgctgcccaaggatctggacacactaagtctattctcaacctccgtaccaacactgagaccaataatccgtgtgccaaaaccatcgtcaagctatttgaagttcaaaaaacagggttcaacattcaggacatggacatagtggcctctgagcacttgctacaccaatcccttgttggcaagcaatccccattccagaacgcctacaacgtcaagggcaatgccaccagtgctaacatcatttaa

SEQ ID NO: 17 - DNA, 656 bp, Artificial Sequence
  Tick vaccine antigen #1 Rhipicephalus appendiculatus salivary gland-associated protein 64P
ggagatcacctgcttgcaaaggacaacgtcctaacacagccgcaaaatgaaagctttcttcgttctttcccttctttcaaccgccgcactgacgaatgcagcaagggctggtcgtcttggaagcgacctggatacatttggaagggtacacggtaacctatatgccggcatcgaaagagctggccctcgtggatacccagggcttaccgcatcgattggaggcgaagtgggtgcacgactcggtggtcgtgccggtgtgggagtgagcagctacggctatggttacccttcatggggctatccgtatggtggatacggtggatacggtggatacggtggatacggtggatatgatcagggttttggctctgcatacggcggctaccccggctactatggctactactatcccagtggctacggtgggggctacggtggtagctacggtggcagctacggtggtagctacacctatcccaacgttcgggcttcagctggtgccgcagcttgagcttctccttcagcgtcacagtaagaaatcatggagcacccgatcgagaaatacagaggttctcaaaagcgtacgggatgccaaccagcaagaaattgcgccgcaaaatgttgagaacaaatacaagttttctgtaaaaaaaaaa

SEQ ID NO: 18 - DNA, 945 bp, Artificial Sequence
  Tick vaccine antigen #2 Rhipicephalus sanguineus acidic ribosomal protein p0
atggtcagggaggataagacgacctggaggagcaactacttcctgcggctggtgcagctgctcgacgagtaccccaagtgcttcatcgtgggcgtcgacaatgtcggttcgaagcagatgcagacgatccgtgtttcgctccgcaagcacgccgtcctgctcatgggcaagaacaccatgatccgcaaggccattcgcggacacctggacaacaacccggccctggaaaagctgttgccgcacatcaagggcaacgtcggtttcgtcttcaccaaggaagacctgacagaggtgcgtgagaagatcattgacaacaaggtgaaggcgcctgcccgtgccggtgccctggcccctctggacgtcatgatcccggcgcagaacaccggcctcggtcccgagaagacctctttcttccaggccctgcagatccccaccaagatctcgaagggtaccattgaaattctcaatgagatccacttgatcaagaaggacgacagggtgggcgcttccgaggccacgcttctcaacatgttgaacatctcgcccttctcgtatggtctgaagattctgcaggtgtacgactccggtaccgtgttctcccccgacattttggacatcacaccagaggacttaagatcagcattcgtcgagggtgtccgcaatgtcgctgctgtatccttgtccatcggatacccgactgttgcatcagtcccacactccattgtcaacggtctcaagaacctcattgccattgccgtggagacagacatcacgttcaaggaggctgaaatggccaaggagtacctcaaggacccgtcaaagttcgctgcagcagcagctccagccgcaggaggtggggcagccgcagccaagccggaggagtcgaagaaggaagaagccaagaaggaggaatccgaagaggaggacgacgacatgggcttctag

SEQ ID NO: 19 - protein, 1688 aa, Artificial Sequence
  CCHFV glycoprotein precursor (CCHF GP-Turkey-kk06)
MPTNIMHTPLVCFILYLQLLCLGGAHGQLNATEHNGTNNTTAPGASQSPKPPMSTTPPHAPESSTIKPTTPISEAEGSGETTSPPNTTQGLSSPETTSERPATTSISTSSTDSTNPTTQMTDNTPTPTVSTSPSSSPSTPSTPQGIHHPARSLLSVSSPKTVTTPTPTSPGEMSSETSSQHSAMSRIPTPHTATRVSTEITNHRTPRQSESSAQQTTPSPMTSPAQSILLMSAAPTAVQDIHPSPTNRSKRNLETEIILTLSQGLKKYYGKILKLLHLTLEQDTEGLLEWCKGNLGSNCDDDFFQKRIEEFFMTGECYFNEVLQFKTLSTLSPTEPSHARLPTAEPFKSYFAKGFLSIDSGYFSAKCYPRSSASGLQLINVTQHPARIAETPGPKTTSLKTINCINLRASVFKEHREVEINVLLPQIAVNLSNCHVVINSHVCDYSLDTDGPVRLPRIYHEGTFIPGTYKIVIDKKNKLNDRCTLVTNCVIKGREVRKGQSVLRQYKTEIKIGKASTGFRKLLSEEPGDDCISRTQLLRTETAEIHDDNYGGPGDKITICNGSTIVDQRLGSELGCYTINRVKSFKLCKNSATGKTCEVDSTPVKCRQGFCLKITQEGRGHVKLSRGSEVVLDACDSSCEVMIPKGTGDILVDCSGGQQHFLKDNLIDLGCPHIPLLGRMAIYICRMSNHPRTTMAFLFWFSFGYVITCIFCKALFYSLIIIGTLGKKIKQYRELKPQTCTICETAPVNAIDAEMHDLNCSYNICPYCASRLTSDGLARHVTQCPKRKEKVEETELYLNLERIPWIVRKLLQVSESTGVALKRSSWLIVLLVLLTVSLSPVQSAPVGHGKTIEIYQTREGFASICLFMLGSILFIVSCLVKGLVDSVSESFFPGLSVCKTCSIGSVNGFEIESHKCYCSLFCCPYCRHCSADREIHQLHLSICKKRKTGSNVMLAVCKRMCFRATIEASRRALLIRSIINTTFVICILTLTICVVSTSAVEMENLPAGTWEREEDLTNFCHQECQVTETECLCPYEALVLRKPLFLDSIVKGMKNLLNSTSLETSLSIEAPWGAINVQSTFKPTVSTANIALSWSSVVHRGNKILVTGRSESIMKLEERTGVSWDLGVEDASESKLLTVSIMDLSQMYSPVFEYLSGDRQVEEWPKATCTGDCPERCGCTSSTCLHKEWSHSRNWRCNPTWCWGVGTGCTCCGVDVKDLFTDHMFVKWKVEYIKTEAIVCVALTSQERQCSLIEAGTRFNLGPVTITLSEPRNIQQKLPPEIITLHPKIEEGFFDLMHVQKVLSASTVCKLQSCTHGIPGDLQVYHIGNLLKGDRVNGHLIHKIESHFNTSWMSWDGCDLDYYCNMGDWPSCTYTGVTQHNHAAFVNLLNIETDYTKTFHFHSKRVTAHGDTPQLDLKARPTYGAGGITVLVEVADMELHTKKVEISGLKFASLACTGCYACSSGISCKVRIHVDEPDELTVHVKSSDPDVVAASTSLMARKLEFGTDSTFKAFSAMPKTSLCFYIVEREYCKSCSEDDTQKCVDTRLEQPQSILIEHKGTIIGKQNDTCTAKASCWLESVKSFFYGLKNMLGSVFGNLFIGILLFLAPFVLLVLFFMFGWKILFCFKCCRRTRGLFKYRHLKDDEETGYRRIIERLNSKKGKNRLLDGDRLADRKIAELFSTKTHIG

SEQ ID NO: 20 - protein, 720 aa, Artificial Sequence
  Ubiquitin -CCHF glycoprotein GC
MQIFVKTLTGKTITLEVEPSDTIENVKAKIQDKEGIPPDQQRLIFAGKQLEDGRTLSDYNIQKESTLHLVLRLRGGFLDSIVKGMKNLLNSTSLETSLSIEAPWGAINVQSTFKPTVSTANIALSWSSVVHRGNKILVTGRSESIMKLEERTGVSWDLGVEDASESKLLTVSIMDLSQMYSPVFEYLSGDRQVEEWPKATCTGDCPERCGCTSSTCLHKEWSHSRNWRCNPTWCWGVGTGCTCCGVDVKDLFTDHMFVKWKVEYIKTEAIVCVALTSQERQCSLIEAGTRFNLGPVTITLSEPRNIQQKLPPEIITLHPKIEEGFFDLMHVQKVLSASTVCKLQSCTHGIPGDLQVYHIGNLLKGDRVNGHLIHKIESHFNTSWMSWDGCDLDYYCNMGDWPSCTYTGVTQHNHAAFVNLLNIETDYTKTFHFHSKRVTAHGDTPQLDLKARPTYGAGGITVLVEVADMELHTKKVEISGLKFASLACTGCYACSSGISCKVRIHVDEPDELTVHVKSSDPDVVAASTSLMARKLEFGTDSTFKAFSAMPKTSLCFYIVEREYCKSCSEDDTQKCVDTRLEQPQSILIEHKGTIIGKQNDTCTAKASCWLESVKSFFYGLKNMLGSVFGNLFIGILLFLAPFVLLVLFFMFGWKILFCFKCCRRTRGLFKYRHLKDDEETGYRRIIERLNSKKGKNRLLDGDRLADRKIAELFSTKTHIG

SEQ ID NO: 21 - protein, 364 aa, Artificial Sequence
  Ubiquitin CCHF Glycoprotein Gn
MQIFVKTLTGKTITLEVEPSDTIENVKAKIQDKEGIPPDQQRLIFAGKQLEDGRTLSDYNIQKESTLHLVLRLRGGSEEPGDDCISRTQLLRTETAEIHDDNYGGPGDKITICNGSTIVDQRLGSELGCYTINRVKSFKLCKNSATGKTCEVDSTPVKCRQGFCLKITQEGRGHVKLSRGSEVVLDACDSSCEVMIPKGTGDILVDCSGGQQHFLKDNLIDLGCPHIPLLGRMAIYICRMSNHPRTTMAFLFWFSFGYVITCIFCKALFYSLIIIGTLGKKIKQYRELKPQTCTICETAPVNAIDAEMHDLNCSYNICPYCASRLTSDGLARHVTQCPKRKEKVEETELYLNLERIPWIVRKLL

SEQ ID NO: 22 - protein, 482 aa, Artificial Sequence
  CCHF Nucleoprotein NP
MENKIEVNNKDEMNRWFEEFKKGNGLVDTFTNSYSFCESVPNLDRFVFQMASATDDAQKDSIYASALVEATKFCAPIYECAWVSSTGIVKKGLEWFEKNAGTIKSWDESYTELKVDVPKIEQLTGYQQAALKWRKDIGFRVNANTAALSNKVLAEYKVPGEIVMSVKEMLSDMIRRRNLILNRGGDENPRGPVSHEHVDWCREFVKGKYIMAFNPPWGDINKSGRSGIALVATGLAKLAETEGKGIFDEAKKTVEALNGYLDKHKDEVDRASADSMITNLLKHIAKAQELYKNSSALRAQSAQIDTAFSSYYWLYKAGVTPETFPTVSQFLFELGKQPRGTKKMKKALLSTPMKWGKKLYELFADDSFQQNRIYMHPAVLTAGRISEMGVCFGTIPVANPDDAAQGSGHTKSILNLRTNTETNNPCAKTIVKLFEVQKTGFNIQDMDIVASEHLLHQSLVGKQSPFQNAYNVKGNATSANII

SEQ ID NO: 23 - DNA, 4029 bp, Artificial Sequence
  pIDV-I
agatcttttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctgtctgacgctcagtggaacgaaaactcacgttaagggattttggtcatgagattatcaaaaaggatcttcacctagatccttttaaattaaaaatgaagttttagcacgtgctattattgaagcacacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaaggcatgcctgctactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcatcgatgcatggtacc

SEQ ID NO: 24 - DNA, 4618 bp, Artificial Sequence
  pIDV-II
agatctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctgtctgacgctcagtggaacgaaaactcacgttaagggattttggtcatgagattatcaaaaaggatcttcacctagatccttttaaattaaaaatgaagttttagcacgtgctattattgaagcacacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaaggcatgcctgctactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcatcgatgcatggtacc

SEQ ID NO: 25 - DNA, 255 bp, Artificial Sequence
  WPRE
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgcc

SEQ ID NO: 26 - DNA, 9693 bp, Artificial Sequence
  pIDV-II-CCHF-GP-Turkey
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctgtctgacgctcagtggaacgaaaactcacgttaagggattttggtcatgagattatcaaaaaggatcttcacctagatccttttaaattaaaaatgaagttttagcacgtgctattattgaagcacacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaaggcatgcctgctactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcatcgatgcatggtacggtaccgcaccatgccaactaacatcacccacaccctgctggtctgcttcatcctgtatctgcagctgctggggagaggcggcgcacatggacagtcaaacgccacagagcacaacggcaccaataccacaaccgcaccaggcacctctcagagccacaagcctctggtgagcacaaccccccctcacacactggagagctccaccatcaagcacacaacccccacctctgagacagagggaagcggagagacaaccccaccacctaacacaacccagggaccttccccaccagaggcaacccctgagcgcccagcaacaaccgccaccagcacaccctccaccgataacacaaatagcacaacccagatgaatgacaacaatcctacctccacaatctccacatctccctctagctccccttctacccctccaacacctcagggcatccaccacccagcacggagcctgctgagcgtgtctagcctgaagaccgccacaaccccaacccccacaagccctggcgagatcagctctgagacaagctcccagcactccgccatgtctcgcaccccaacactgcacacaaccacacaggtgagcaccgagtccacaaaccactccaccccaaggcagtctgagtctagcgcccagcctaccacaccttccccaatgacatctccagcccagagcatcctgcccatgtctgccgcccctaccgccatccagaatatccaccccagccctacaaaccggtccaagagaaatctggaggtggagatcatcctgaccctgtcccagggcctgaagaagtactatggcaagatcctgaagctgctgcacctgacactggaggaggataccgagggcctgctggagtggtgtaagagaaacctgggctcctcttgcgacgatgacttctttcagaagaggatcgaggagttctttgtgaccggcgagggctactttaatgaggtgctgcagttcaagaccctgtctacactgagccccacagagcctagccacgccaagctgccaaccgtggagcccttcaagtcctattttgccaagggcttcctgtccatcgactctggctacttttccgccaagtgttatccacgcagctccacatctggcctgcagctgatcaacgtgacccagcacccagcaaggatcgcagagacaccaggacccaagaccacatctctgaagaccatcaactgcatcaatctgagggccagcgtgttcaaggagcaccgcgagatcgagatcaatgtgctgctgccacagatcgccgtgaacctgagcaattgtcacgccgtgatcaagtctcacgtgtgcgattacagcctggataccgacggccctgtgagactgccacacatctaccacgagggcacattcatccccggcacctataagatcgtgatcgataagaagaacaagctgaatgacaggtgtatcctggtgaccaactgcgtgatcaagggaagggaggtgcgcaagggacagtccgtgctgagacagtataagaccgagatcaagatcggcaaggccagcacaggctccaggaagctgctgtccgaggagcctggcgatgactgcatctctaggacccagctgctgaggaccgagacagcagagatccacgatgacaactacggcggcccaggcgataagatcacaatctgtaatggaagcaccatcgtggaccagcgcctgggatccgagctgggctgctataccatcaaccgggtgaagagctttaagctgtgcgagaattccgccaccggcaagacatgcgagatcgacagcacccctgtgaagtgtagacagggcttctgcctgaagatcacacaggagggccggggccacgtgaagctgtctagaggcagcgaggtggtgctggacgtgtgcgactctagctgcgaagtgatgatcccaaagggcaccggcgatatcctggtggactgctccggaggacagcagcactttctgaaggataacctgatcgacctgggatgtccacacgtgccactgctgggaagaatggccatctacatctgccggatgtccaatcaccccagaaccacaatggccttcctgttttggttctcttttggctacgtgatcacctgcatcttttgtaaggccctgttctatagcctgatcatcatcggcacactgggcaagaagttcaagcagtatagggagctgaagccccagacctgcacaatctgtgagacagcccctgtgaacgccatcgatgccgagatgcacgacctgaactgttcctacaatatctgcccctattgtgcatccaggctgacctctgatggcctggcaagacacgtgcctcagtgcccaaagaggaaggagaaggtggaggagacagagctgtacctgaatctggagaggatcccttggatcgtgcgcaagctgctgcaggtgagcgagtccaccggagtggccctgaagagatcctcttggctgatcgtgctgctggtgctgctgacagtgtctctgagcccagtgcagagcgccccagtgggacacggcaagaccatcgagacatatcagaccagggagggctttacctccatctgtctgttcatgctgggctccatcctgttcatcgtgtcttgcctggtgaagggcctggtggattccgtgtctgacagcttctttcccggcctgagcgtgtgcaagacctgttccatcggctctatcaacggctttgagatcgagagccacaagtgctactgttccctgttctgctgtccttattgccggcactgttccgccgacagagagatccaccagctgcacctgtctatctgcaagaagagaaagaccggcagcaacgtgatgctggccgtgtgcaagaggatgtgctttcgcgccacaatcgaggcctctcggagagccctgctgatcaggagcatcatcaataccacattcgtgatctgtatcctgaccctgacaatctgcgtggtgtccacctctgccgtggagatggagaatctgccagcaggcacatgggagagggaggaggatctgaccaacttttgtcaccaggagtgccaggtgaccgagacagagtgcctgtgcccatacgaggccctggtgctgaggaagcctctgttcctggacagcatcgtgaagggcatgaagaacctgctgaatagcacatccctggagacaagcctgagcatcgaggcaccatggggagccatcaacgtgcagtctacctttaagcccacagtgagcaccgccaatatcgccctgtcctggagctccatcgagcaccgcggcaacaagatcctggtgaccggccggtccgagtctatcatgaagctggaggagaggacaggcgtgagctgggatctgggagtggaggacgcaagcgagtccaagctgctgaccgtgagcatcatggacctgagccagatgtactcccccgtgttcgagtatctgtccggcgatagacaggtggaggagtggccaaaggccacctgtacaggcgactgccccgagaggtgcggctgcacatctagcacctgtctgcacaaggagtggcctcacagccggaactggagatgtaatccaacctggtgctggggagtgggcacaggatgcacctgctgtggcgtggatgtgaaggacctgtttacagatcacatgttcgtgaagtggaaggtggagtacatcaagaccgaggccatcgtgtgcgtggagctgacatctcaggagagacagtgcagcctgatcgaggccggcaccaggttcaatctgggcccagtgaccatcacactgagcgagccccgcaacatccagcagaagctgccccctgagatcatcacactgcacccaaaggtggaggagggcttctttgacctgatgcacgtgcagaaggtgctgtctgccagcaccgtgtgcaagctgcagtcctgcacccacggaatcccaggcgatctgcaggtgtaccacatcggcaacctgctgaagggcgaccgggtgaatggccacctgatccacaagatcgagccacactttaataccagctggatgtcctgggatggctgtgatctggactactattgcaacatgggcgactggcccagctgcacctacacaggcgtgacccagcacaatcacgccgccttcgtgaacctgctgaatatcgagacagattataccaagacattccactttcactccaagcgcgtgacagcccacggcgatacccctcagctggacctgaaggcccggccaacatacggagcaggagagatcaccgtgctggtggaggtggccgacatggagctgcacaccaagaaggtggagatcagcggcctgaagtttgcctctctggcctgcacaggctgttatgcctgctcctctggcatcagctgcaaggtgcgcatccacgtggatgagcctgacgagctgaccgtgcacgtgaagagctccgatccagacgtggtggcagcatccacatctctgaccgcacggaagctggagtttggcacagacagcaccttcaaggccttttccgccatgcctaagacctctctgtgcttctacatcgtggagaaggagtattgtaagtcttgcaacgaggatgacacacagaagtgcgtggataccaagctggagcagccacagagcatcctgatcgagcacaagggcaccatcatcggcaagcagaatgacacctgtacagccaaggcctcctgctggctggagtctgtgaagagcttcttttacggcctgaagaacatgctgggcagcgtgttcggcaatttctttatcggcatcctgctgtttctggcccccttcgtgctgctggtgctgttctttatgtttggctggaagatcctgttctgctttaagtgctgtaggcgcaccaggggcctgttcaagtaccgccacctgaaggatgacgaggagacaggctataagcggatcatcgagagactgaacaataagaagggcaagaacagactgctggacggcgagagactggcagaccggaaaatcgcagagctgtttagtaccaaaactcacatcgggtgatga

SEQ ID NO: 27 - DNA, 5070 bp, Artificial Sequence
  CCHF-GP-Turkey
atgccaactaacatcacccacaccctgctggtctgcttcatcctgtatctgcagctgctggggagaggcggcgcacatggacagtcaaacgccacagagcacaacggcaccaataccacaaccgcaccaggcacctctcagagccacaagcctctggtgagcacaaccccccctcacacactggagagctccaccatcaagcacacaacccccacctctgagacagagggaagcggagagacaaccccaccacctaacacaacccagggaccttccccaccagaggcaacccctgagcgcccagcaacaaccgccaccagcacaccctccaccgataacacaaatagcacaacccagatgaatgacaacaatcctacctccacaatctccacatctccctctagctccccttctacccctccaacacctcagggcatccaccacccagcacggagcctgctgagcgtgtctagcctgaagaccgccacaaccccaacccccacaagccctggcgagatcagctctgagacaagctcccagcactccgccatgtctcgcaccccaacactgcacacaaccacacaggtgagcaccgagtccacaaaccactccaccccaaggcagtctgagtctagcgcccagcctaccacaccttccccaatgacatctccagcccagagcatcctgcccatgtctgccgcccctaccgccatccagaatatccaccccagccctacaaaccggtccaagagaaatctggaggtggagatcatcctgaccctgtcccagggcctgaagaagtactatggcaagatcctgaagctgctgcacctgacactggaggaggataccgagggcctgctggagtggtgtaagagaaacctgggctcctcttgcgacgatgacttctttcagaagaggatcgaggagttctttgtgaccggcgagggctactttaatgaggtgctgcagttcaagaccctgtctacactgagccccacagagcctagccacgccaagctgccaaccgtggagcccttcaagtcctattttgccaagggcttcctgtccatcgactctggctacttttccgccaagtgttatccacgcagctccacatctggcctgcagctgatcaacgtgacccagcacccagcaaggatcgcagagacaccaggacccaagaccacatctctgaagaccatcaactgcatcaatctgagggccagcgtgttcaaggagcaccgcgagatcgagatcaatgtgctgctgccacagatcgccgtgaacctgagcaattgtcacgccgtgatcaagtctcacgtgtgcgattacagcctggataccgacggccctgtgagactgccacacatctaccacgagggcacattcatccccggcacctataagatcgtgatcgataagaagaacaagctgaatgacaggtgtatcctggtgaccaactgcgtgatcaagggaagggaggtgcgcaagggacagtccgtgctgagacagtataagaccgagatcaagatcggcaaggccagcacaggctccaggaagctgctgtccgaggagcctggcgatgactgcatctctaggacccagctgctgaggaccgagacagcagagatccacgatgacaactacggcggcccaggcgataagatcacaatctgtaatggaagcaccatcgtggaccagcgcctgggatccgagctgggctgctataccatcaaccgggtgaagagctttaagctgtgcgagaattccgccaccggcaagacatgcgagatcgacagcacccctgtgaagtgtagacagggcttctgcctgaagatcacacaggagggccggggccacgtgaagctgtctagaggcagcgaggtggtgctggacgtgtgcgactctagctgcgaagtgatgatcccaaagggcaccggcgatatcctggtggactgctccggaggacagcagcactttctgaaggataacctgatcgacctgggatgtccacacgtgccactgctgggaagaatggccatctacatctgccggatgtccaatcaccccagaaccacaatggccttcctgttttggttctcttttggctacgtgatcacctgcatcttttgtaaggccctgttctatagcctgatcatcatcggcacactgggcaagaagttcaagcagtatagggagctgaagccccagacctgcacaatctgtgagacagcccctgtgaacgccatcgatgccgagatgcacgacctgaactgttcctacaatatctgcccctattgtgcatccaggctgacctctgatggcctggcaagacacgtgcctcagtgcccaaagaggaaggagaaggtggaggagacagagctgtacctgaatctggagaggatcccttggatcgtgcgcaagctgctgcaggtgagcgagtccaccggagtggccctgaagagatcctcttggctgatcgtgctgctggtgctgctgacagtgtctctgagcccagtgcagagcgccccagtgggacacggcaagaccatcgagacatatcagaccagggagggctttacctccatctgtctgttcatgctgggctccatcctgttcatcgtgtcttgcctggtgaagggcctggtggattccgtgtctgacagcttctttcccggcctgagcgtgtgcaagacctgttccatcggctctatcaacggctttgagatcgagagccacaagtgctactgttccctgttctgctgtccttattgccggcactgttccgccgacagagagatccaccagctgcacctgtctatctgcaagaagagaaagaccggcagcaacgtgatgctggccgtgtgcaagaggatgtgctttcgcgccacaatcgaggcctctcggagagccctgctgatcaggagcatcatcaataccacattcgtgatctgtatcctgaccctgacaatctgcgtggtgtccacctctgccgtggagatggagaatctgccagcaggcacatgggagagggaggaggatctgaccaacttttgtcaccaggagtgccaggtgaccgagacagagtgcctgtgcccatacgaggccctggtgctgaggaagcctctgttcctggacagcatcgtgaagggcatgaagaacctgctgaatagcacatccctggagacaagcctgagcatcgaggcaccatggggagccatcaacgtgcagtctacctttaagcccacagtgagcaccgccaatatcgccctgtcctggagctccatcgagcaccgcggcaacaagatcctggtgaccggccggtccgagtctatcatgaagctggaggagaggacaggcgtgagctgggatctgggagtggaggacgcaagcgagtccaagctgctgaccgtgagcatcatggacctgagccagatgtactcccccgtgttcgagtatctgtccggcgatagacaggtggaggagtggccaaaggccacctgtacaggcgactgccccgagaggtgcggctgcacatctagcacctgtctgcacaaggagtggcctcacagccggaactggagatgtaatccaacctggtgctggggagtgggcacaggatgcacctgctgtggcgtggatgtgaaggacctgtttacagatcacatgttcgtgaagtggaaggtggagtacatcaagaccgaggccatcgtgtgcgtggagctgacatctcaggagagacagtgcagcctgatcgaggccggcaccaggttcaatctgggcccagtgaccatcacactgagcgagccccgcaacatccagcagaagctgccccctgagatcatcacactgcacccaaaggtggaggagggcttctttgacctgatgcacgtgcagaaggtgctgtctgccagcaccgtgtgcaagctgcagtcctgcacccacggaatcccaggcgatctgcaggtgtaccacatcggcaacctgctgaagggcgaccgggtgaatggccacctgatccacaagatcgagccacactttaataccagctggatgtcctgggatggctgtgatctggactactattgcaacatgggcgactggcccagctgcacctacacaggcgtgacccagcacaatcacgccgccttcgtgaacctgctgaatatcgagacagattataccaagacattccactttcactccaagcgcgtgacagcccacggcgatacccctcagctggacctgaaggcccggccaacatacggagcaggagagatcaccgtgctggtggaggtggccgacatggagctgcacaccaagaaggtggagatcagcggcctgaagtttgcctctctggcctgcacaggctgttatgcctgctcctctggcatcagctgcaaggtgcgcatccacgtggatgagcctgacgagctgaccgtgcacgtgaagagctccgatccagacgtggtggcagcatccacatctctgaccgcacggaagctggagtttggcacagacagcaccttcaaggccttttccgccatgcctaagacctctctgtgcttctacatcgtggagaaggagtattgtaagtcttgcaacgaggatgacacacagaagtgcgtggataccaagctggagcagccacagagcatcctgatcgagcacaagggcaccatcatcggcaagcagaatgacacctgtacagccaaggcctcctgctggctggagtctgtgaagagcttcttttacggcctgaagaacatgctgggcagcgtgttcggcaatttctttatcggcatcctgctgtttctggcccccttcgtgctgctggtgctgttctttatgtttggctggaagatcctgttctgctttaagtgctgtaggcgcaccaggggcctgttcaagtaccgccacctgaaggatgacgaggagacaggctataagcggatcatcgagagactgaacaataagaagggcaagaacagactgctggacggcgagagactggcagaccggaaaatcgcagagctgtttagtaccaaaactcacatcgggtgatga

SEQ ID NO: 28 - protein, 1688 aa, Artificial Sequence
  CCHF-GP-Turkey
MPTNITHTLLVCFILYLQLLGRGGAHGQSNATEHNGTNTTTAPGTSQSHKPLVSTTPPHTLESSTIKHTTPTSETEGSGETTPPPNTTQGPSPPEATPERPATTATSTPSTDNTNSTTQMNDNNPTSTISTSPSSSPSTPPTPQGIHHPARSLLSVSSLKTATTPTPTSPGEISSETSSQHSAMSRTPTLHTTTQVSTESTNHSTPRQSESSAQPTTPSPMTSPAQSILPMSAAPTAIQNIHPSPTNRSKRNLEVEIILTLSQGLKKYYGKILKLLHLTLEEDTEGLLEWCKRNLGSSCDDDFFQKRIEEFFVTGEGYFNEVLQFKTLSTLSPTEPSHAKLPTVEPFKSYFAKGFLSIDSGYFSAKCYPRSSTSGLQLINVTQHPARIAETPGPKTTSLKTINCINLRASVFKEHREIEINVLLPQIAVNLSNCHAVIKSHVCDYSLDTDGPVRLPHIYHEGTFIPGTYKIVIDKKNKLNDRCILVTNCVIKGREVRKGQSVLRQYKTEIKIGKASTGSRKLLSEEPGDDCISRTQLLRTETAEIHDDNYGGPGDKITICNGSTIVDQRLGSELGCYTINRVKSFKLCENSATGKTCEIDSTPVKCRQGFCLKITQEGRGHVKLSRGSEVVLDVCDSSCEVMIPKGTGDILVDCSGGQQHFLKDNLIDLGCPHVPLLGRMAIYICRMSNHPRTTMAFLFWFSFGYVITCIFCKALFYSLIIIGTLGKKFKQYRELKPQTCTICETAPVNAIDAEMHDLNCSYNICPYCASRLTSDGLARHVPQCPKRKEKVEETELYLNLERIPWIVRKLLQVSESTGVALKRSSWLIVLLVLLTVSLSPVQSAPVGHGKTIETYQTREGFTSICLFMLGSILFIVSCLVKGLVDSVSDSFFPGLSVCKTCSIGSINGFEIESHKCYCSLFCCPYCRHCSADREIHQLHLSICKKRKTGSNVMLAVCKRMCFRATIEASRRALLIRSIINTTFVICILTLTICVVSTSAVEMENLPAGTWEREEDLTNFCHQECQVTETECLCPYEALVLRKPLFLDSIVKGMKNLLNSTSLETSLSIEAPWGAINVQSTFKPTVSTANIALSWSSIEHRGNKILVTGRSESIMKLEERTGVSWDLGVEDASESKLLTVSIMDLSQMYSPVFEYLSGDRQVEEWPKATCTGDCPERCGCTSSTCLHKEWPHSRNWRCNPTWCWGVGTGCTCCGVDVKDLFTDHMFVKWKVEYIKTEAIVCVELTSQERQCSLIEAGTRFNLGPVTITLSEPRNIQQKLPPEIITLHPKVEEGFFDLMHVQKVLSASTVCKLQSCTHGIPGDLQVYHIGNLLKGDRVNGHLIHKIEPHFNTSWMSWDGCDLDYYCNMGDWPSCTYTGVTQHNHAAFVNLLNIETDYTKTFHFHSKRVTAHGDTPQLDLKARPTYGAGEITVLVEVADMELHTKKVEISGLKFASLACTGCYACSSGISCKVRIHVDEPDELTVHVKSSDPDVVAASTSLTARKLEFGTDSTFKAFSAMPKTSLCFYIVEKEYCKSCNEDDTQKCVDTKLEQPQSILIEHKGTIIGKQNDTCTAKASCWLESVKSFFYGLKNMLGSVFGNFFIGILLFLAPFVLLVLFFMFGWKILFCFKCCRRTRGLFKYRHLKDDEETGYKRIIERLNNKKGKNRLLDGERLADRKIAELFSTKTHIG

SEQ ID NO: 29 - DNA, 6856 bp, Artificial Sequence
  pIDV-II-Ebola-GP-M06
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctgtctgacgctcagtggaacgaaaactcacgttaagggattttggtcatgagattatcaaaaaggatcttcacctagatccttttaaattaaaaatgaagttttagcacgtgctattattgaagcacacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaaggcatgcctgctactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcatcgatgcatggtacggatccgccaccatgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttgtaattgcagttatcgctttattctgtatatgcaaatttgtcttttagtttttcttcagattgcttcatggaaaagctcagcctcaaatcaatgaaaccaggatttaattatatggattacttgaatctaagattacttgacaaatgataatataatacactggagctttaaacatagccaatgtgattctaactcctttaaactcacagttaatcataaacaaggtttgaggtaccgagctcgaattc

SEQ ID NO: 30 - DNA, 2232 bp, Artificial Sequence
  Ebola-GP-M06
atgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttgtaattgcagttatcgctttattctgtatatgcaaatttgtcttttagtttttcttcagattgcttcatggaaaagctcagcctcaaatcaatgaaaccaggatttaattatatggattacttgaatctaagattacttgacaaatgataatataatacactggagctttaaacatagccaatgtgattctaactcctttaaactcacagttaatcataaacaaggtttgaggtaccgagctcgaattc

SEQ ID NO: 31 - protein, 676 aa, Artificial Sequence
  Ebola-GP-M06
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVVIAVIALFCICKFVF

SEQ ID NO: 32 - DNA, 6664 bp, Artificial Sequence
  pIDV-II-HA86-p0
gaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcatcgatgcatggtaccatgtgctctccgccactgtttgttggtgcgatccttctgatcgtggggtgcgctgggcaggttctgcgagctcaaccgactagcagtgtgtgtagcgattttggaaaacaattttgccaaaacgccgagtgtgaggtcatcccagggcgcgaggacgattttgtctgccgctgccctaaagatgatatgtattacaacgccgctgagaagcagtgtgagtataagagaacctgcaagacggttgaatgttcttatggcaactgtgtgcagattagtcccgggcgcaccgactgcgggtgccaaggagtggacacgttgaccctcaaatgtggcatccaggagtggtatgctaacgagtgcggtcgccgcggtggaacggctgttcgccgcactgatggttttctcggggcacgctgtgactgtggtgagtgggggaagatgtcaaagggaccaaatggcaaatgcgtgcccacaacttgtattcgccccgacctgacatgcaaggatttgtgcgagaaaaatttgcttggcaaagatacccgctgttgtcaaggatggaatccgacagactgctctgttgttccaccagaagatacatattgcagcccgggttctattaagggcgaggatggcaagtgtattgatgcgtgtacaactaaggaagcactgttgctctgtaaggatgggtgtatcaaggggcaaaagcccggaaaagcctataagtgcatttgtccccatggttacgagatagcggaggacggcatcacttgcaagcgcgttcctgggatagtcgattgtaccgaagagcagaaggcggcttgtcttcccggccagcagtgtagagtgcataaggagaatagcgtgtgtgaatgtccatccgaccaacagttgcttgacggaaaatgcgcgagtgaatgcgttgacaaccggtgccatgaaaatttcaccgattgtggagtttatatgaacaaacagggatgctactgcccgtggacaacccgaaagccacctggaggagttgaaattagcaggtgcatgcttaatgagtattactacacagtctcatttacgcccaacatctctcttaactccgaccattgtgaatggtacgaaaagcgagtccttgaggcaatgaggacagcgataggtgtcgaagtcttcaaagtggagattatgaactgtacacaggacataatggctaggctgatcgcatcaagaccccttagtaatcacgtcttgaataagcttcaagcctgtgaacatccggttggagatttctgtatgctgtatccgaagctccccataaaaaaagggtctgccacagggatcgaggaagagaatctctgcgaatccctgctgaagaaccaagagaaggcgtataaaggtgaaaataaatgcgttaaggtggatgacttctattggttccaatgtgctgatggatacagggcggtcagggatgttaccagaggccgcctcaggagatccgtctgtaaggcaggagtgtcttgcactgataaagagcaacttgattgtgcgaataaggggcagatatgcgtctttgagaatgaaaaacccaattgtcaatgcccgccggatacggtgcctggtcaggccggctgtgcagcccggacgacttgcaatcctaaggaaattagggaatgtgaggacaaaaagaaagaatgtgtctatcgggatcaaaaggcagaatgccagtgtcccgaagggacagttgattacggtcaagggtgttctggggggccggtggaagcgtcctgtactgaggaaagcattgccgagtgtcgcagctctggcaagagatgcgccatcgaaaatggccgaccaatatgcaaagagacttccggtgttgttacggccgaggccacgacgacagaagcaacaaaagcagatccggaccccggaaaatcaggtggtgtggccgcggcaggagggggtgccgccgcagccaagccggaggagtcgaagaaggaagaagccaagaagtggtgcgaatgcagatctcatcatcatcatcaccatcaccaccaccactagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatctgtctgacgctcagtggaacgaaaactcacgttaagggattttggtcatgagattatcaaaaaggatcttcacctagatccttttaaattaaaaatgaagttttagcacgtgctattattgaagcacacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaaggcatgcctgctactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtc

SEQ ID NO: 33 - DNA, 2052 bp, Artificial Sequence
  HA86-p0
atgtgctctccgccactgtttgttggtgcgatccttctgatcgtggggtgcgctgggcaggttctgcgagctcaaccgactagcagtgtgtgtagcgattttggaaaacaattttgccaaaacgccgagtgtgaggtcatcccagggcgcgaggacgattttgtctgccgctgccctaaagatgatatgtattacaacgccgctgagaagcagtgtgagtataagagaacctgcaagacggttgaatgttcttatggcaactgtgtgcagattagtcccgggcgcaccgactgcgggtgccaaggagtggacacgttgaccctcaaatgtggcatccaggagtggtatgctaacgagtgcggtcgccgcggtggaacggctgttcgccgcactgatggttttctcggggcacgctgtgactgtggtgagtgggggaagatgtcaaagggaccaaatggcaaatgcgtgcccacaacttgtattcgccccgacctgacatgcaaggatttgtgcgagaaaaatttgcttggcaaagatacccgctgttgtcaaggatggaatccgacagactgctctgttgttccaccagaagatacatattgcagcccgggttctattaagggcgaggatggcaagtgtattgatgcgtgtacaactaaggaagcactgttgctctgtaaggatgggtgtatcaaggggcaaaagcccggaaaagcctataagtgcatttgtccccatggttacgagatagcggaggacggcatcacttgcaagcgcgttcctgggatagtcgattgtaccgaagagcagaaggcggcttgtcttcccggccagcagtgtagagtgcataaggagaatagcgtgtgtgaatgtccatccgaccaacagttgcttgacggaaaatgcgcgagtgaatgcgttgacaaccggtgccatgaaaatttcaccgattgtggagtttatatgaacaaacagggatgctactgcccgtggacaacccgaaagccacctggaggagttgaaattagcaggtgcatgcttaatgagtattactacacagtctcatttacgcccaacatctctcttaactccgaccattgtgaatggtacgaaaagcgagtccttgaggcaatgaggacagcgataggtgtcgaagtcttcaaagtggagattatgaactgtacacaggacataatggctaggctgatcgcatcaagaccccttagtaatcacgtcttgaataagcttcaagcctgtgaacatccggttggagatttctgtatgctgtatccgaagctccccataaaaaaagggtctgccacagggatcgaggaagagaatctctgcgaatccctgctgaagaaccaagagaaggcgtataaaggtgaaaataaatgcgttaaggtggatgacttctattggttccaatgtgctgatggatacagggcggtcagggatgttaccagaggccgcctcaggagatccgtctgtaaggcaggagtgtcttgcactgataaagagcaacttgattgtgcgaataaggggcagatatgcgtctttgagaatgaaaaacccaattgtcaatgcccgccggatacggtgcctggtcaggccggctgtgcagcccggacgacttgcaatcctaaggaaattagggaatgtgaggacaaaaagaaagaatgtgtctatcgggatcaaaaggcagaatgccagtgtcccgaagggacagttgattacggtcaagggtgttctggggggccggtggaagcgtcctgtactgaggaaagcattgccgagtgtcgcagctctggcaagagatgcgccatcgaaaatggccgaccaatatgcaaagagacttccggtgttgttacggccgaggccacgacgacagaagcaacaaaagcagatccggaccccggaaaatcaggtggtgtggccgcggcaggagggggtgccgccgcagccaagccggaggagtcgaagaaggaagaagccaagaagtggtgcgaatgcagatctcatcatcatcatcaccatcaccaccaccactag

SEQ ID NO: 34 - protein, 683 aa, Artificial Sequence
  HA86-p0
MCSPPLFVGAILLIVGCAGQVLRAQPTSSVCSDFGKQFCQNAECEVIPGREDDFVCRCPKDDMYYNAAEKQCEYKRTCKTVECSYGNCVQISPGRTDCGCQGVDTLTLKCGIQEWYANECGRRGGTAVRRTDGFLGARCDCGEWGKMSKGPNGKCVPTTCIRPDLTCKDLCEKNLLGKDTRCCQGWNPTDCSVVPPEDTYCSPGSIKGEDGKCIDACTTKEALLLCKDGCIKGQKPGKAYKCICPHGYEIAEDGITCKRVPGIVDCTEEQKAACLPGQQCRVHKENSVCECPSDQQLLDGKCASECVDNRCHENFTDCGVYMNKQGCYCPWTTRKPPGGVEISRCMLNEYYYTVSFTPNISLNSDHCEWYEKRVLEAMRTAIGVEVFKVEIMNCTQDIMARLIASRPLSNHVLNKLQACEHPVGDFCMLYPKLPIKKGSATGIEEENLCESLLKNQEKAYKGENKCVKVDDFYWFQCADGYRAVRDVTRGRLRRSVCKAGVSCTDKEQLDCANKGQICVFENEKPNCQCPPDTVPGQAGCAARTTCNPKEIRECEDKKKECVYRDQKAECQCPEGTVDYGQGCSGGPVEASCTEESIAECRSSGKRCAIENGRPICKETSGVVTAEATTTEATKADPDPGKSGGVAAAGGGAAAAKPEESKKEEAKKWCECRSHHHHHHHHHH

SEQ ID NO: 35 - DNA, 50 bp, Artificial Sequence
  Probe  binding sequence
ccgcccagtagaaggcatgcctgctactagttattaatagtaatcaatta

(19) United States
(12) Patent Application Publication
(10) Pub. No.: US 2021/0220463 A1
(43) Pub. Date: Jul. 22, 2021

(54) VECTORS FOR DNA VACCINATION

(71) Applicant: UNIVERSITÉ LAVAL, Québec (CA)

(72) Inventors: Gary KOBINGER, Québec (CA); George BABUADZE, Toronto (CA); Trina RACINE, Winnipeg (CA)

(21) Appl. No.: 17/054,308

(22) PCT Filed: May 21, 2019
(86) PCT No.: PCT/CA2019/050686

§ 371 Date: Nov. 10, 2020

Related U.S. Application Data
(60) Provisional application No. 62/673,387, filed on May 18, 2018.

(51) Int. Cl. A61K 39/12; C12N 15/86; C07K 14/005; A61P 31/14; A61P 37/04

(52) U.S. Cl. CPC A61K 39/12; C12N 15/86; C07K 14/005; A61K 2039/53; A61P 37/04; C12N 2760/12022; A61P 31/14

(57) ABSTRACT

The present disclosure provides vectors that allow efficient expression of transgenes. The vector of the present disclosure may be used to express proteins or peptides of interest into a host's cells and to trigger an immune response towards an antigenic portion of the proteins or peptides in a mammal. The vectors may be used for experimental research, for pre-clinical or clinical application. The vectors disclosed herein induce both cell-mediated and humoral immune responses and may be used in DNA vaccination.

### TECHNICAL FIELD

The present disclosure relates to vectors that allow efficient expression of transgenes. The vectors may be used for experimental research, for pre-clinical or clinical applications and more particularly, for DNA vaccination.

### BACKGROUND

DNA vaccines have recently deserved high interest. DNA vaccination relies on administration of DNA vectors encoding an antigen, or multiple antigens, for which an immune response is sought into a host. DNA vectors include elements that allow expression of the protein by the host's cells, and includes a strong promoter, a poly-adenylation signal and sites where the DNA sequence of the transgene is inserted. Vectors also contain elements for their replication and expansion within microorganisms. DNA vectors can be produced in high quantities over a short period of time and as such they represent a valuable approach in response to outbreaks of new pathogens. In comparison with recombinant proteins, whole-pathogen, or subunit vaccines, their method of manufacturing are relatively cost-effective and they can be supplied without the use of a cold chain system.

DNA vaccines have been tested in animal disease models of infection, cancer, allergy and autoimmune disease. They generate a strong humoral and cellular immune response that has generally been found to protect animals from the disease.

Several DNA vaccines have been tested in human clinical trials including DNA vaccines for Influenza virus, Dengue Virus, Venezuelan Equine Encephalitis Virus, HIV, Hepatitis B Virus, Plasmodium Falciparum Malaria, Herpes Simplex, Zika virus etc. (Tebas, P. et al., N Engl J Med, 2017 (DOI: 10.1056/NEJMoa1708120); Gaudinski, M. R. et al., Lancet, 391:552-62, 2018).

The potency of DNA vaccines has been improved with the advent of new delivery approaches and improvements in vector design.

A number of technical improvements are being explored, such as gene optimization strategies, improved RNA structural design, novel formulations and immune adjuvants, and various effective delivery approaches. DNA based vaccines offers a number of potential advantages over traditional approaches, including the stimulation of both B- and T-cell responses, improved stability and the absence of infectious agent.

Several DNA vectors are under development for a variety of infectious agents including influenza virus, hepatitis B virus, human immunodeficiency virus, rabies virus, lymphocytic chorio-meningitis virus, malarial parasites and mycoplasmas. However, in spite of good humoral or cellular responses the protection from disease in animals has been obtained only in some cases.

There remains a need for improving the efficiency of DNA vaccination. The inventors have generated vectors that show efficient transgene expression. These vectors may be used for experimental research, for pre-clinical or clinical application and more particularly, for DNA vaccination.

In the present study, high-expression vectors are used to generate recombinant candidate vaccines expressing three different virus glycoproteins and one tick antigen.

### SUMMARY

In a first aspect, the present disclosure relates to vectors for expressing transgenes encoding complete protein(s), protein fragment(s) or peptide(s). The vector of the present disclosure may be used to express proteins or peptides of interest into a host's cells and to trigger an immune response towards an antigenic portion of the proteins or peptides in a mammal.

In a further aspect the present disclosure relates to a vector which may comprise a CMV enhancer, a chicken beta actin promoter, a site for cloning a transgene, a polyadenylation signal and a neomycin/kanamycin expression cassette in reverse orientation or opposite direction.

The vector may further comprise a chimeric intron at the 3′-end of the chicken beta actin promoter, an ampicillin resistance promoter, and/or a 3′ flanking region of rabbit β-Globin at the 3′-end of the polyadenylation signal.

In another aspect, the present disclosure relates to a vector having a nucleic acid sequence at least 90% identical, at least 95% identical or that is identical to the sequence set forth in SEQ ID NO.:1.

In yet another aspect, the present disclosure relates to a vector comprising a transgene. The vector may thus comprise a gene encoding a protein(s) or peptide(s) of interest, such as for example, antigens from a pathogen, from a tumor (i.e., a tumor-specific antigen), from an allergen or a protein suitable for treatment of an autoimmune disease. The vector may also comprise a gene that may act as an adjuvant.

Exemplary embodiments of transgenes include: genes encoding antigens from virus(es), bacteria or parasite(s) and/or a combination thereof. In another exemplary embodiment, the transgene may be a gene encoding a therapeutic protein. In yet another exemplary embodiment, the transgene may be a gene encoding an adjuvant molecule.

Circular forms or linear forms of the vectors are also encompassed by the present disclosure.

In accordance with the present disclosure the vector may be used for research applications, for pre-clinical or for clinical applications.

### BRIEF DESCRIPTION OF THE DRAWINGS

FIG. 1: schematic illustrating the different elements contained in the vector; the circular form (FIG. 1A) and a linearized form (FIG. 1B) are represented.

FIG. 2: schematic of the pCAGGS-eGFP used as a positive control.

FIG. 3: histogram representing eGFP expression by fluorescent activated cell sorter (FACS). Vero E6 cells were transfected in triplicate with either pIDV-eGFP, pVAX1-eGFP, or pCAGGS-eGFP using Lipofectamine 2000 (control cells received only Lipofectamine 2000). eGFP expression was analyzed 24 hours after transfection. The average (and standard deviation) eGFP expression of two replicate experiments is presented.

FIG. 4: histogram representing eGFP expression by fluorescent activated cell sorter (FACS), 24 hours post-transfection in VeroE6 cells. The graph shows the average and standard deviation of the eGFP expression of 4 different DNA vectors in transfected cells.

FIG. 5: picture of a Western blot under non-reduced conditions with anti-CCHFV monoclonal antibody −11E7 (used against the Gn protein of entire GP) as shown by a single protein expression of approximately 75 kDa; a) pIDV-II-CCHF-GP-Turkey (SEQ ID NO:26), b) pVAX1-CCHF-GP-Turkey and c) pCAGGS-CCHF-GP-Turkey Transfection in 293-LTV cells. 6 well plates. 300.000 cells/well, 5 μg DNA/well. Cell lyses with non-reduced condition lyses buffer. Western blot: 24 h after transfection. Proteins were quantified and ≈15 ug cell lysate+loading buffer was loaded into the blotting gel. Primary antibody: monoclonal anti-GP CCHF 11E7 dilution − 1/2000. Secondary 1:20000 of secondary anti −a-Tubulin antibody and anti-mouse IgG, dilution − 1/10000. CCHF GP of approximately 75 kDa (arrow), confirming recombinant protein expression. A loading control (lane 2) of 50 kDa shows an equal amount of loaded proteins.

FIG. 6: picture of a Western blot a) pIDV-II-Ebola-GP-M06 (SEQ ID NO:29), b) pCAGGS-Ebola-GP-M06 and c) pVAX1-Ebola-GP-M06; Transfection in 293-LTV cells. 6 well plates. 300.000 cells/well, 5 μg DNA/well. Cell lyses with xTractor lysis buffer (BD). Western blot: 24 h after transfection. Proteins were quantified and ≈15 ug cell lysate+10 ul loading buffer was loaded into the blotting gel. Primary antibody: monoclonal anti-4F3 mouse anti EBOV GPd™ mAb dilution − 1/2000. Secondary 1:20000 of secondary anti −a-Tubulin antibody and anti-mouse IgG, dilution − 1/10000.

FIG. 7: picture of a Western blot a) pIDV-II plasmid encoding HIV envelope , b) pVAX1 plasmid encoding HIV envelope and c) pCAGGS plasmid encoding HIV envelope .Transfection in 293-LTV cells. 6 well plates. 300.000 cells/well, 5 μg DNA/well. Cell lyses with xTractor lysis buffer (BD). Western blot: 24h after transfection. Proteins were quantified and ≈15 ug cell lysate+10 ul loading buffer was loaded into the blotting gel. Primary antibody: monoclonal anti-ID6 mouse anti EBOV GPd™ mAb dilution − 1/2000. Secondary 1:20000 of secondary anti −a-Tubulin antibody and anti-mouse IgG, dilution − 1/10000

FIG. 8: picture of a Western blot a) pIDV-II-HA86-p0 (SEQ ID NO:32), b) pVAX1-HA86-p0 and c) pCAGGS- HA86-p0 Transfection in 293-LTV cells. 6 well plates. 300.000 cells/well, 5 μg DNA/well. Cell lyses with xTractor lysis buffer (BD). Western blot: 24 h after transfection. Proteins were quantified and ≈15 ug cell lysate+10 ul loading buffer was loaded into the blotting gel. Primary antibody: His Tag mAb-mouse dilution − 1/2500. Secondary 1:20000 of secondary anti −a-Tubulin antibody and Anti-Mouse IgG (H+L) Antibody, Human Serum Adsorbed and Peroxidase-Labeled − 1/20000.

FIGS. 9a-f: alignment of pIDV-I and pIDV-II sequence.

FIG. 10: graph showing IFN-g ELISpot responses from Balb/c mice immunized with pIDV-II-CCHF-GP-Turkey or pVAX1-CCHF-GP-Turkey. Asterisks indicate statistically significant differences (****, p<0.005).

FIG. 11: graph showing Ebola glycoprotein (GP)-specific T-cell responses from mice vaccinated with pIDV-II-EboV-GP-M06 or pVAX1-EboV-GP-M06 as assessed by the IFN-γ ELISpot. Asterisks indicate statistically significant differences (**, p<0.005; *, p<0.05).

FIG. 12: graph showing CCHFV-specific IgG following immunization with pIDV-II-CCHF-GP-Turkey or with pVAX1-CCHF-GP-Turkey. *Two-way ANOVA, confidence intervals were set to 95%., P-value=<0.0001.

FIG. 13: graph showing Ebola glycoprotein (GP) specific IgG titers following immunization with pIDV-II-Ebov-GP-M06 compared to pVAX1-Ebov-GP-M06.

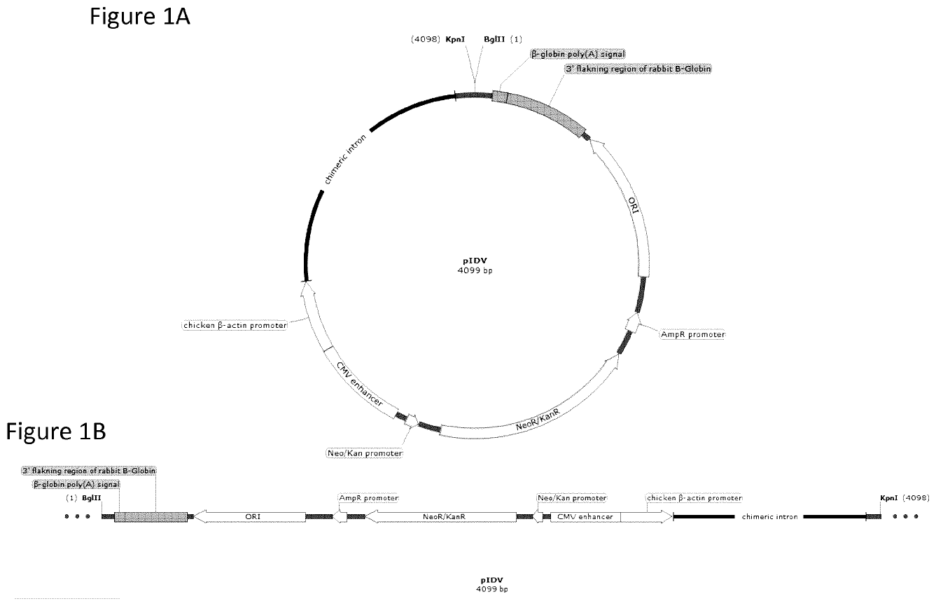

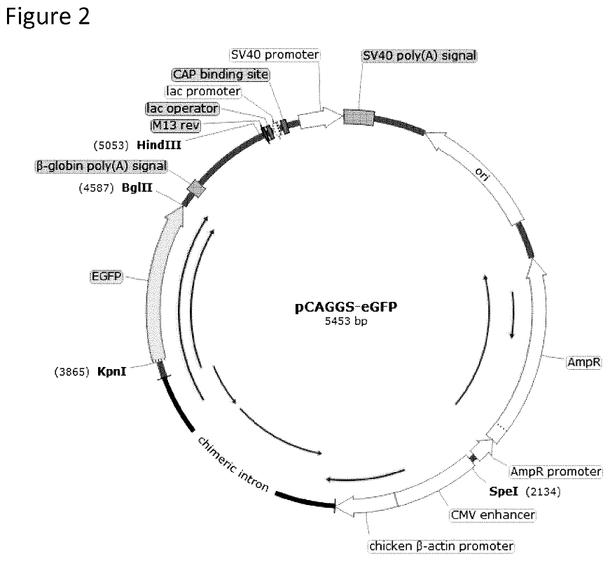

### DETAILED DESCRIPTION

The present disclosure provides in one aspect thereof vectors for expression of transgenes. The vectors of the present disclosure may be used for DNA vaccination.

In accordance with the present disclosure, the vector may comprise for example, the sequence set forth in SEQ ID NO.1 or a sequence at least 80%, at least 85%, at least 90%, at least 95% or at least 99% identical to SEQ ID NO:1.

In accordance with the present disclosure, the vector may comprise for example, the sequence set forth in SEQ ID NO.23 or a sequence at least 80%, at least 85%, at least 90%, at least 95% or at least 99% identical to SEQ ID NO:23.

In accordance with the present disclosure, the vector may comprise for example, the sequence set forth in SEQ ID NO.24 or a sequence at least 80%, at least 85%, at least 90%, at least 95% or at least 99% identical to SEQ ID NO:24.

It is to be understood herein that the percentage of identity does not take into account the presence of transgene.

The vector comprises elements that are arranged in a manner to increase expression of the transgene(s). For example, the vector may comprise a CMV enhancer, a chicken beta actin promoter, a site for cloning a transgene, a polyadenylation signal and a neomycin/kanamycin expression cassette in reverse orientation or opposite direction.

The vector of the present disclosure may be used to express complete protein(s), protein fragment(s) or peptide(s) for experimental research, for pre-clinical or clinical applications.

In accordance with the present disclosure, the vector may comprise a) a CMV enhancer having a sequence that is at least 90% identical, at least 95% identical, at least 99% identical or that is identical to the sequence set forth in SEQ ID NO.:2, b) a chicken beta actin promoter having a sequence that is at least 90% identical, at least 95% identical, at least 99% identical or that is identical to the sequence set forth in SEQ ID NO.:3, c) a polyadenylation signal having a sequence that is at least 90% identical, at least 95% identical, at least 99% identical or that is identical to the sequence set forth in SEQ ID NO.:4, d) a 3′ flanking region of rabbit β-Globin having a sequence that is at least 90% identical, at least 95% identical, at least 99% identical or that is identical to the sequence set forth in SEQ ID NO.:5, e) an origin of replication having a sequence that is at least 90% identical, at least 95% identical, at least 99% identical or that is identical to the sequence set forth in SEQ ID NO.:6, f) optionally an ampicillin resistance promoter having a sequence that is at least 90% identical, at least 95% identical, at least 99% identical or that is identical to the sequence set forth in SEQ ID NO.: 7, g) a neomycin/kanamycin resistance gene having a sequence that is at least 90% identical, at least 95% identical, at least 99% identical or that is identical to the sequence set forth in SEQ ID NO.:8, and/or h) a NeoR/KanR promoter having a sequence that is at least 90% identical, at least 95% identical, at least 99% identical or that is identical to the sequence set forth in SEQ ID NO.:9.

The vector may further comprise posttranscriptional regulatory elements. In accordance with the present disclosure, the posttranscriptional regulatory element may be from a virus such as for example and without limitation, from Hepatitis B virus or from Woodchuck Hepatitis virus.

In accordance with the present invention, the posttranscriptional regulatory element may be a Woodchuck Hepatitis Virus Posttranscriptional Regulatory Element (WPRE) and may have a sequence as set forth in SEQ ID NO:25 or a sequence at least 80% identical, at least 85% identical, at least 90% identical, at least 95% identical or at least 99% identical to SEQ ID NO:25.

In accordance with an aspect of the present disclosure, the AmpR promoter may be absent from the vector.

More particularly, the vector of the present disclosure may have a nucleotide sequence that is at least 90% identical, at least 95% identical or that is identical to the sequence set forth in SEQ ID NO.:1.

In an exemplary embodiment, the sequence of the vector may be as set forth in SEQ ID NO.:1 (pIDV).

In a further exemplary embodiment, the sequence of the vector may be as set forth in SEQ ID NO:23 (pIDV-I).

Yet in a further exemplary embodiment, the sequence of the vector may be as set forth in SEQ ID NO:24 (pIDV-II).

A nucleic acid sequence encoding a given antigen(s) may be cloned into the pIDV, pIDV-I or pIDV-II vector and administered to a host in order to induce an immune response against the antigen(s). The present disclosure therefore encompasses vectors comprising a nucleic acid sequence encoding an antigen or antigens.

### Antigens

Antigens selected for expression in the pIDV, pIDV-I or PIDV-II vector may be from a pathogen, from a tumor (a tumor specific antigen) from an allergen, etc.

The present disclosure provides in a further aspect thereof, transgenes that may able to trigger an immune response.

In accordance with the present disclosure, the transgene may encode a Crimean Congo Hemorrhagic Fever virus protein such as for example, a CCFH glycoprotein and/or nucleoprotein.

In an exemplary embodiment, the transgene may be able to encode the protein set forth in SEQ ID NO: 20 (with or without the ubiquitin portion), SEQ ID NO: 21 (with or without the ubiquitin portion), SEQ ID NO: 22 or SEQ ID NO: 28.

In an exemplary embodiment, the transgene may have the sequence set forth in SEQ ID NO: 13 or a sequence at least 80% identical, at least 85% identical, at least 90% identical, at least 95% identical or at least 99% identical.

In a further exemplary embodiment, the transgene may have the sequence set forth in SEQ ID NO: 14 at least 80% identical, at least 85% identical, at least 90% identical, at least 95% identical or at least 99% identical.

In another exemplary embodiment, the transgene may have the sequence set forth in SEQ ID NO: 15 at least 80% identical, at least 85% identical, at least 90% identical, at least 95% identical or at least 99% identical.

In another exemplary embodiment, the transgene may have the sequence set forth in SEQ ID NO: 16 at least 80% identical, at least 85% identical, at least 90% identical, at least 95% identical or at least 99% identical.

In a further exemplary embodiment, the transgene may have the sequence set forth in SEQ ID NO: 27 at least 80% identical, at least 85% identical, at least 90% identical, at least 95% identical or at least 99% identical.

In accordance with the present disclosure, the transgene may encode an Ebola protein, such as for example, an Ebola glycoprotein.

In an exemplary embodiment, the transgene may be able to encode the protein set forth in SEQ ID NO:31 (with or without the M06 portion).

The transgene may have, for example, the sequence set forth in SEQ ID NO:30 at least 80% identical, at least 85% identical, at least 90% identical, at least 95% identical or at least 99% identical.

Further in accordance with the present disclosure, the transgene may encode an HIV protein such as for example, an HIV envelope and/or gag protein.

In accordance with the present disclosure, the transgene may encode a tick antigen.

In an exemplary embodiment, the transgene may be able to encode the protein set forth in SEQ ID NO:35 (with or without the p0 portion).

The transgene may have, for example, the sequence set forth in SEQ ID NO:33 at least 80% identical, at least 85% identical, at least 90% identical, at least 95% identical or at least 99% identical.

It is to be understood that the transgene is not limited to the above and may include other transgenes from pathogens and/or encoding tumor-specific antigens.

It is also to be understood herein that the transgene may be designed so as to have a sufficient level of identity with different strains or isolates of the same pathogen.

The present disclosure also provides for the antigen encoded by any of the transgene disclosed herein. Such antigen may be formulated in pharmaceutical composition for therapeutic use including without limitation for eliciting an immune response and/or for vaccination. Such antigen may also be used as tools in research and development including for example and without limitation in electrophoresis, ELISA assays and the like.

The antigen may be monovalent or multivalent (e.g., a multi-chain protein composed of several antigens from a single pathogen, from multiple pathogens, from different strains, isolates, serotype of a given pathogen). The antigen may also be a consensus sequence derived from the amino acid sequence of different strains, isolates, or serotypes of a given pathogen.

Generally, the specific strain(s), isolate(s) or serotype(s) of pathogen used for generating the vaccine of the present disclosure may be selected from the strain(s), isolate(s) or serotype(s) that is(are) prevalent in a given population. In the case of new outbreaks, the gene expressing the antigen or antigens may be sequenced and cloned into the vector of the present disclosure using methods known in the art involving for example, amplification by polymerase chain reaction, use of restriction enzymes, ligation, transformation of bacteria, sequencing, etc.

Exemplary embodiments of antigens include without limitation, viral antigens from Retroviridae (HIV, HTLV), Flaviviridae (e.g., Zika, Hepatitis C, West Nile, Dengue, Yellow fever, Japanese encephalitis, tick-borne encephalitis, Saint Louis encephalitis, Alkhurma hemorrhagic fever virus, Kyasanur Forest Disease virus, Omsk hemorrhagic fever virus etc.), Togaviridae (e.g., Chikungunya, Rubella virus), Picornaviridae (Hepatitis A, Polio virus, Enterovirus (EV71)), Caliciviridae (Norwalk virus, Sapporo virus), Astroviridae, Coronaviridae (e.g., Middle East Respiratory syndrome coronavirus, Severe acute Respiratory Syndrome coronavirus, etc.), Rhabdoviridae (rabies), Filoviridae (Ebola virus, Marburg virus), Paramixoviridae (Nipah virus, Hendra virus, Measles virus, Mumps virus, Respiratory syncytial virus), Orthomixoviridae (Influenza virus H1N1, H3N2, H5N1, H7N9), Bunyaviridae (Rift Valley Fever Disease virus, Crimean-Congo hemorrhagic fever virus, Hantaan, Dobrava, Saarema, Seoul and Puumala viruses, Hanta virus), Arenaviridae (Lassa virus, Junin virus, Guanarito virus, Lujo virus, Sbia virus, Machupo virus, Whitewater Arroyo virus, Chapare virus, Lymphocytic choriomeningitis virus), Reoviridae (rotavirus), Papovaviridae (human papilloma viruses), Adenoviridae, Parvoviridae, Herpesviridae (Herpes simplex virus, varicella-zoster virus, Epstein-Barr virus, cytomegalovirus), Poxviridae (smallpox virus, vaccinia virus), Hepadnaviridae (Hepatitis B).

Exemplary embodiments of antigens include without limitation, bacterial antigens from Salmonella Typhi, Salmonella Parathyphi, Yersinia pestis, Vibrio cholera, Corynebacterium diphtheria, Haemophilus influenza type B, Neisseria meningitidis, Bordetella pertussis, Streptococcus pneumoniae, Clostridium tetani, Clostridium difficile, Mycobacterium tuberculosis, Campylobacter jejuni, enterotoxigenic Escherichia coli, Streptococcus agalactiae (group B), Streptococcus pneumoniae, Streptococcus pyrogenes, Salmonella enterica, Shigella, Staphylococcus aureus.

Exemplary embodiments of antigen also include without limitation, parasite antigens from Plasmodium (Plasmodium falciparum, Plasmodium vivax, Plasmodium ovale, Plasmodium malariae, Plasmodium Know lesi), Trypanosome (Trypanosoma cruzi), Necator americanus, Leishmania, Schistosoma haematobium, Schistosoma mansoni, H. anatolicumanatolicum, H. dromedarii, Rhipicephalus sanguineus, etc.

Exemplary embodiments of tumor antigens include without limitation; 707 alanine proline-AFP (707-AP), alpha (α)-fetoprotein (AFP), adenocarcinoma antigen recognized by T cells 4 (ART-4), B antigen; β-catenin/mutated (BAGE), breakpoint cluster region-Abelson (Bcr-abl), CTL-recognized antigen on melanoma (CAMEL), carcinoembryonic antigen peptide-1 (CAP-1), caspase-8 (CASP-8), cell-division-cycle 27 mutated (CDC27m), cycline-dependent kinase 4 mutated (CDK4/m), carcino-embryonic antigen (CEA), cancer testis antigen (CT), cyclophilin B (Cyp-B), differentiation antigen melanoma (DAM), elongation factor 2 mutated (ELF2M), Ets variant gene ⁶/ₐcute myeloid leukemia 1 gene ETS (ETV6-AML1), glycoprotein 250 (G250), G antigen (GAGE), N-acetylglucosaminyltransferase V (GnT-V), glycoprotein 100 kDa (Gp100), helicose antigen (HAGE), human epidermal receptor-2/neurological (HER-2/neu), arginine (R) to isoleucine (I) exchange at residue 170 of the α-helix of the α2-domain in the HLA-A2 gene (HLA-A*0201-R1701), human papilloma virus E7 (HPV-E7), heat shock protein 702 mutated (HSP70-2M), human signet ring tumor-2 (HST-2), human telomerase reverse transcriptase (hTERT or hTRT), intestinal carboxyl esterase (iCE), KIAA0205, L antigen (LAGE), low-density lipid receptor/GDP-L-fucose: β-D-galactosidase 2-α-L-fucosyltransferase (LDLR/FUT), melanoma antigen (MAGE), melanoma antigen recognized by T cells-1/melanoma antigen A (MART-1/Melan-A), melanocortin 1 receptor (MC1R), myosin mutated (Myosin/m), mucin 1 (MUC1), melanoma ubiquitous mutated 1, 2, 3 (MUM-1, -2, -3), NA cDNA clone of patient M88 (NA88-A), New York-esophagus 1 (NY-ESO-1), protein 15 (P15), protein of 190 kDa ber-abl (p190 minor bcr-abl), promyelocytic leukaemia/retinoic acid receptor α (Pml/RARα), preferentially expressed antigen of melanoma (PRAME), prostate-specific antigen (PSA), prostate-specific membrane antigen (PSMA), renal antigen (RAGE), renal ubiquitous 1 or 2 (RU1 or RU2), sarcoma antigen (SAGE), squamous antigen rejecting tumor 1 or 3 (SART-1 or SART-3), translocation Ets-family leukemia/acute myeloid leukemia 1 (TEL/AML1), triosephosphate isomerase mutated (TPI/m), tyrosinase related protein 1 or gp75 (TRP-1), tyrosinase related protein 2 (TRP-2), TRP-2/intron 2 (TRP-2/INT2), Wilms' tumor gene (WT1).

In order to generate a stronger immune response in a host, it may be desirable to select a surface antigen of a pathogen, such as glycoproteins of viruses or suitable fragments thereof (e.g., HIV gp160 or gp120, Ebola virus glycoprotein (e.g., from the Zaire species), Nipah virus glycoprotein, Zika virus envelope and/or pre-membrane M (prM), Lassa fever virus glycoprotein, Crimean Congo Hemorrhagic Fever virus glycoprotein). However, a vaccine for a given pathogen may include other types of antigens. For example, structural proteins such as the viral capsid, nucleocapsid, matrix, including HIV gag, CCHF nucleocapsid. etc.

For veterinary purposes, the pathogen may be selected amongst animal-specific pathogens or amongst pathogens causing zoonotic diseases. Examples of veterinary vaccines are provided for example, in Roth, J. A., 2011 (Procedia in Vaccinology 5: 127-136, 2011) and Redding L. and D. B. and Weiner, 2009 (Expert Rev. Vaccines 8(9), 1251-1276, 2009). Licensed products for animal vaccination include preventative vaccines for West Nile virus in horses and infectious haematopoietic necrosis virus in fish, a therapeutic cancer vaccine for dogs, and a growth hormone gene therapy to increase litter survival in breeding pig sows.

Exemplary embodiments of antigens for DNA vaccination, devices and methods for their administration or for enhancing their delivery are disclosed in Larocca, R. A. et al. (Nature, 536:474, 2016), WO/2017/190147, WO/2017/136758, WO/2017/117273, WO/2017/117508, WO/2017/117251, WO/2016/153995, WO/2016/154071, WO/2016/123285, WO/2016/089862, WO/2016/054003, WO/2015/103602, WO/2015/089492, WO/2015/081155, WO/2015/073291, WO/2015/054012, WO/2015/023461, WO/2014/165291, WO/2014/151279, WO/2014/150835, WO/2014/150835, WO/2014/152121, WO/2014/144885, W0/2014/145951, WO/2014/144731, WO/2014/145038, WO/2014/144786, WO/2014/093886, WO/2014/093894, WO/2014/093897, WO/2014/047286, WO/2013/158792, WO/2013/155441, WO/2013/066427, WO/2013/062507, WO/2013/05541, WO/2013/055326, WO/2013/055420, WO/2012/065164, WO/2012/047679, WO/2011/137221, WO/2011/109406, WO/2011/109399, WO/2011/054011, WO/2010/050939, WO/2009/091578, WO/2008/148010, WO/2008/143988, WO/2004/004825, US2018011714, the entire content of which is incorporated herein by reference.

Antigens that have been tested as DNA vaccines disclosed in the art may be suitable for expression into the pIDV, pIDV-I or pIDV-II vector. Examples of suitable antigens may be found for example in the DNAVaxDB database (Racz et al. BMC Bioinformatics 2014, 15(Suppl 4):S2).

### Vaccines

The present disclosure provides in yet a further aspect thereof DNA vaccines.

The DNA vaccine may comprise a pIDV, pIDV-I or pIDV-II vector or a variant at least 80% identical, at least 85% identical, at least 90% identical, at least 95% identical or at least 99% identical and a transgene.

In accordance with the present disclosure the DNA vaccine may comprise a pIDV, pIDV-I or pIDV-II vector and a transgene encoding a Crimean Congo Hemorrhagic Fever virus protein such as for example, a CCFH glycoprotein and/or nucleoprotein.

In accordance with the present disclosure the DNA vaccine may comprise a pIDV, pIDV-I or pIDV-II vector and a transgene having the sequence set forth in SEQ ID NO: 13.

Further in accordance with the present disclosure, the DNA vaccine may comprise a pIDV, pIDV-I or pIDV-II vector and a transgene having the sequence set forth in SEQ ID NO: 14.

Also in accordance with the present disclosure, the DNA vaccine may comprise a pIDV, pIDV-I or pIDV-II vector and a transgene having the sequence set forth in SEQ ID NO: 15.

In accordance with the present disclosure, the DNA vaccine may comprise a pIDV, pIDV-I or pIDV-II vector and a transgene having the sequence set forth in SEQ ID NO: 16.

Further in accordance with the present disclosure, the DNA vaccine may comprise a pIDV, pIDV-I or pIDV-II vector and a transgene having the sequence set forth in SEQ ID NO: 27.

In a particular embodiment the DNA vaccine may comprise the pIDV-II vector (SEQ ID NO:23) and a transgene selected from the group consisting of SEQ ID NO:13, 14, 15, 16 or 27.

Exemplary embodiment of DNA vaccine for Crimean Congo Hemorrhagic Fever virus include for example and without limitation the plasmid set forth in SEQ ID NO:26. Variants having at least 80% identical, at least 85% identical, at least 90% identical, at least 95% identical or at least 99% identity with SEQ ID NO:26 are also encompassed.

In accordance with the present disclosure, the DNA vaccine may comprise a pIDV, pIDV-I or pIDV-II vector and a transgene encoding an Ebola protein, such as for example, an Ebola glycoprotein.

For example, the DNA vaccine may comprise a pIDV, pIDV-I or pIDV-II vector and a transgene having the sequence set forth in SEQ ID NO:30.

In a particular embodiment the DNA vaccine may comprise the pIDV-II vector (SEQ ID NO:23) and the transgene having the sequence set forth in SEQ ID NO:30.

Exemplary embodiments of DNA vaccine for Ebola virus include, for example and without limitation, the plasmid set forth in SEQ ID NO:29. Variants having at least 80% identical, at least 85% identical, at least 90% identical, at least 95% identical or at least 99% identity with SEQ ID NO:29 are also encompassed.

In accordance with the present disclosure, the DNA vaccine may comprise a pIDV, pIDV-I or pIDV-II vector and a transgene encoding an HIV protein such as for example, an HIV envelope and/or gag protein. In a particular embodiment the DNA vaccine may comprise the pIDV-II vector (SEQ ID NO:23) and the transgene able to encode an HIV envelope and/or gag protein.

In accordance with the present disclosure, the DNA vaccine may comprise a pIDV, pIDV-I or pIDV-II vector and transgene encoding a tick antigen.

For example, the DNA vaccine may comprise a pIDV, pIDV-I or pIDV-II vector and a transgene encoding a tick antigen and having the sequence set forth in SEQ ID NO:33. In a particular embodiment the DNA vaccine may comprise the pIDV-II vector (SEQ ID NO:23) and a transgene having the sequence set forth in SEQ ID NO:33.

In an exemplary embodiment, the DNA vaccine for tick may include, for example and without limitation, the plasmid set forth in SEQ ID NO:32. Variants having at least 80% identical, at least 85% identical, at least 90% identical, at least 95% identical or at least 99% identity with SEQ ID NO:32 are also encompassed.

In accordance with an embodiment of the disclosure, the DNA vaccine may comprise a pharmaceutically acceptable carrier. The vaccine may further comprise an adjuvant.

The DNA vaccine of the present disclosure may comprise a mixture of different vectors (e.g., pIDV-II) each encoding a different antigen either from the same pathogen or from different pathogens.

### Method of Manufacturing

Methods for manufacturing DNA vectors for vaccination are known in the art and are based on guidance from the FDA (USA Food and Drug Administration. Guidance for Industry: Considerations for Plasmid DNA Vaccines for Infectious Disease Indications. Rockville, Md., USA: 2007) or the EMA (European Medicines Agency. Note for Guidance on the Quality, Preclinical and Clinical Aspects of Gene Transfer Medicinal Products. London, UK: 2001. CPMP/BWP/3088/99; Presence of the Antibiotic Resistance Marker Gene nptII in GM Plants and Food and Feed Uses. London, UK: 2007. EMEA/CVMP/56937/2007).

Exemplary methods of manufacturing are reviewed in Williams J. A., 2013 (Vaccines, 1(3): 225-249, 2013). Processes for high-scale production and purification are also disclosed in Carnes, A. E. and J. A. Williams, 2007 (Recent Patents on Biotechnology, 1:151-66, 2007).

Plasmid DNA production is typically performed in endA (DNA-specific endonuclease I), recA (DNA recombination) deficient E. coli K12 strains such as DH5a, DH5, DH1, XL1Blue, GT115, JM108, DH10B, or endA, recA engineered derivatives of alternative strains such as MG1655, or BL21.

Transformed bacteria are fermented using for example, fed-batch fermentation processes. Clinical grade DNA vector can be obtained by various methods (e.g., HyperGRO™) through service providers such as Aldevron, Eurogentec and VGXI.

DNA vectors are then purified to remove bacterial debris and impurities (RNA, genomic DNA, endotoxins) and formulated with a suitable carrier (for research purposes) or pharmaceutical carrier (for pre-clinical or clinical applications).

### Pharmaceutical Compositions

DNA vectors of the present disclosure may be administered as a pharmaceutical composition, which may comprise for example, the DNA vector(s) and a pharmaceutically acceptable carrier.

The pharmaceutical composition may comprise a single DNA vector species encoding one or more antigens. The one or more antigens may be, for example, from the same pathogen, from closely-related pathogens, or from different pathogens.

Alternatively, the pharmaceutical composition may comprise a mixture of DNA vector species (multiple DNA vector species) each encoding different antigens. For example, the different antigens may be from the same pathogen, from closely-related pathogens, or from different pathogens.

The pharmaceutical composition may further comprise additional elements for increasing uptake of the DNA vector by the cells, its transport in the nucleic, expression of the transgene, secretion, immune response, etc.

The pharmaceutical composition may comprise for example, adjuvant molecule(s). The adjuvant molecule(s) may be encoded by the DNA vector that encodes the antigen or by another DNA vector. Encoded adjuvant molecule(s) may include DNA- or RNA-based adjuvant (CpG oligonucleotides, immunostimulatory RNA, etc.) or protein-based immunomodulators.

The adjuvant molecule(s) may be co-administered with the DNA vectors.

Adjuvants include, but are not limited to, mineral salts (e.g., AlK(SO₄)2, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs), CpG oligonucleotides, immunostimulatory RNA, poly IC or poly AU acids, saponins such as QS21, QS17, and QS7 (U.S. Pat. Nos. 5,057,540; 5,650,398; 6,524,584; 6,645,495), monophosphoryl lipid A, such as 3-de-O-acylated monophosphoryl lipid A (3D-MPL), imiquimod, lipid-polymer matrix (ENABL™ adjuvant), Emulsigen-D™ etc.

A pIDV, pIDV-I or pIDV-II vector expressing an antigen may be formulated for administration by injection (e.g., intramuscular, intradermal, transdermal, subcutaneously) or for mucosal administration (oral, intranasal).

In accordance with the present disclosure, the pharmaceutical composition may be formulated into nanoparticles.

### Method of Administration

The DNA vectors of the present disclosure may be administered to humans or to animals (non-human primates, cattle, rabbits, mice, rats, sheep, goats, horses, birds, poultry, fish, etc.). The DNA vector may thus be used as a vaccine in order to trigger an immune response against an antigen of interest in a human or animal.

The pIDV, pIDV-I or pIDV-II vector expressing the antigen of interest may be administered alone (e.g., as a single dose or in multiple doses) or co-administered with a recombinant antigen, with a viral vaccine (live (e.g., replication competent or not), attenuated, inactivated, etc.), with suitable therapy for modulating or boosting the host's immune response such as for example, adjuvants, immunomodulators (cytokine, chemokines, checkpoint inhibitors, etc.), etc. A pIDV, pIDV-I or pIDV-II vector expressing the antigen of interest may also be co-administered with a plasmid encoding molecules that may act as adjuvant. In accordance with the present disclosure, such adjuvant molecules may also be encoded by the pIDV, pIDV-I or pIDV-II vector (e.g., CpG motifs, cytokine, chemokines, etc.).

In some instances, the pIDV, pIDV-I or pIDV-II vector may be administered first (for priming) and the recombinant antigen or viral vaccine may be administered subsequently (as a boost), or vice versa.

The pIDV, pIDV-I or pIDV-II vector expressing an antigen may be administered by injection intramuscularly, intradermally, transdermally, subcutaneously, to the mucosa (oral, intranasal), etc.

In accordance with the present disclosure, the vaccine may be administered by a physical delivery system including via electroporation, a needleless pressure-based delivery system, particle bombardment, etc.

Following administration, the host's immune response towards the antigen may be assessed using methods known. In some instances, the level of antibodies against the antigen may be measured by ELISA assay or by other methods known by a person skilled in the art. The cellular immune response towards the antigen may be assessed by ELISPOT or by other methods known by a person skilled in the art.

In the case of pre-clinical studies in animals, the level of protection against the pathogen may be determined by challenge experiments where the pathogen is administered to the animal and the animal's health or survival is assessed. The level of protection conferred by the vaccine expressing a tumor antigen may be determined by tumor shrinkage or inhibition of tumor growth in animal models carrying the tumor.

### Definitions

As used herein the terms “vector” and “plasmid” are used interchangeably.

As used herein the term “vector backbone” refers to the vector portion of a given vector into which the sequence of a transgene has been cloned.

It is to be understood herein that the term “single DNA vector species” refers to a composition of vectors where each vector of the composition has the same nucleic acid sequence as the others. The term “multiple DNA vector species” refers to a composition comprising one or more “single DNA vector species”.

The term “transgene” refers to a gene encoding the protein(s) or peptide(s) of interest inserted in the vector of the present disclosure.

As used herein the term “opposite direction” with respect to a gene(s) of the DNA vector of the present disclosure refers to an orientation that is reversed in comparison with the other elements of the DNA vector.

As used herein, the term “reverse orientation” refers to the orientation of a gene(s) of the DNA vector of the present disclosure that is reversed in comparison with a similar gene(s) found in the pVAX1™ vector of reference.

As used herein the terms “human virus” or “human viruses” refer to a virus(es) capable of infecting humans. It is to be understood herein that a “human virus” encompasses animal viruses that infect humans. It is also understood herein that the “human virus” of the present disclosure encompasses viruses causing diseases in humans.

As used herein the term “90% sequence identity”, includes all values contained within and including 90% to 100%, such as 91%, 92%, 92,5%, 95%, 96.8%, 99%, 100%. Likely, the term “at least 75% identical” includes all values contained within and including 75% to 100%.

Generally, the degree of similarity and identity between two sequences is determined using the Blast2 sequence program (Tatiana A. Tatusova, Thomas L. Madden (1999), “Blast 2 sequences—a new tool for comparing protein and nucleotide sequences”, FEMS Microbiol Lett. 174:247-250) using default settings, i.e., meagablast program (see NCBI Handout Series|BLAST homepage & search pages|Last Update Sep. 8, 2016).

It is to be understood herein that the nucleic acid sequences encoding protein(s) or peptide(s) of interest may be codon-optimized. The term “codon-optimized” refers to a sequence for which a codon has been changed for another codon encoding the same amino acid but that is preferred or that performs better in a given organism (increases expression, minimize secondary structures in RNA etc.). “Codon-optimized” sequences may be obtained, using publicly available softwares or via service providers including GenScript (OptimumGene™, U.S. Pat. No. 8,326,547).

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), etc.

The term “treatment” for purposes of this disclosure refers to both therapeutic treatment and prophylactic or preventative measures, wherein the object is to slow down (lessen) the targeted pathologic condition or disorder. Those in need of treatment include those already with the disorder as well as those prone to have the disorder or those in whom the disorder is to be prevented.

All patents, patent applications, and publications referred to herein are incorporated by reference in their entirety.

### EXAMPLE 1−Construction of the pIDV Vector

The pIDV vector was designed to allow easy insertion and subsequent high expression of exogenous genes in a wide variety of mammalian cells.

In Silico Design of pIDV

The pVAX1™ sequence (SEQ ID NO.:10) was uploaded in Geneious™ software and modifications were designed. The first modification removed nucleotides 32-1054 from pVAX1™, which contains the CMV promoter, the T7 promoter, the multiple cloning site and the bGH polyA terminator.

A number of additional modifications were made in silico using the Geneious software and then the circularized plasmid was ordered from GenScript™ and tested. This plasmid represents the first generation of pIDV.

However, we discovered that subsequent modifications further improved the vector including reversion the ORI/Neo/Kan cassette. The pIDV vector of the present disclosure (SEQ ID NO.:1) comprises a CMV enhancer, a chicken β-actin promoter, an intron, a β-globin poly(A) signal and a 3′ flanking region all originating from pCAGGS (U.S. Pat. No. 8,663,981 and described in Richardson J. et al. Enhanced protection against Ebola virus mediated by an improved Adenovirus-based vaccine, PLOS One, 4(4), e5308, 2009) and also contains a Neomycin/Kanamycin promoter, a Neomycin/Kanamycin resistance gene, an Ampicillin promoter and the Ori originating from the pVAX1™ sequence obtained online (SEQ ID NO.:10).

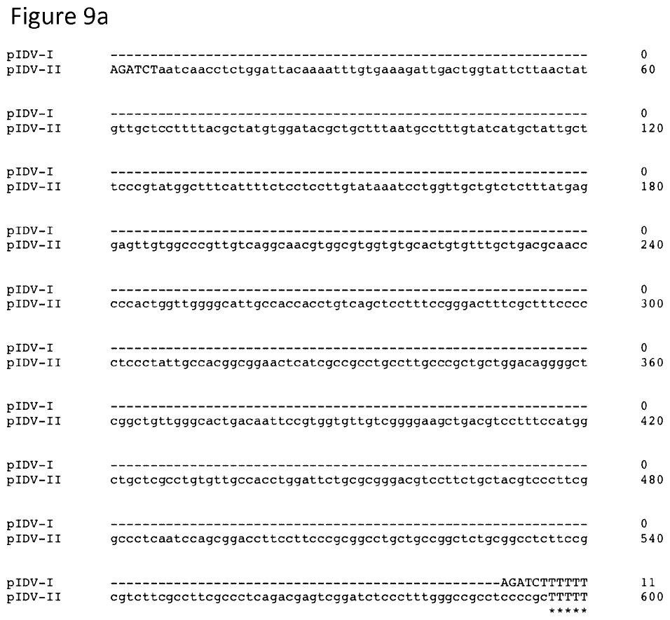

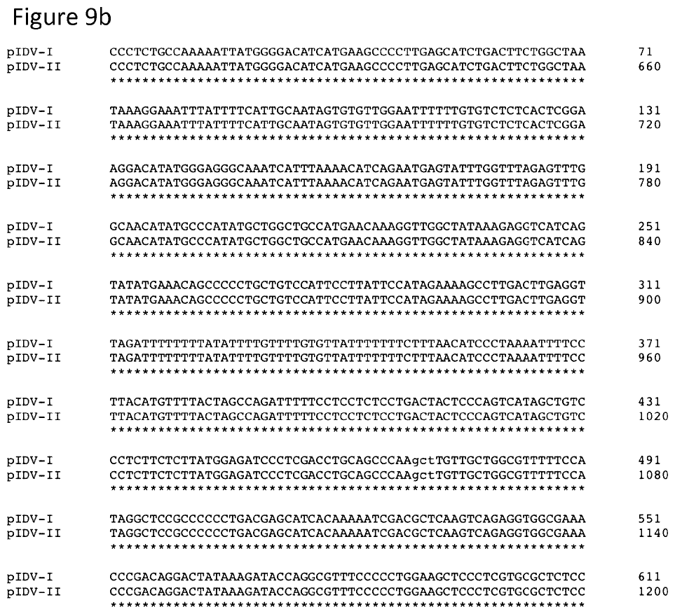

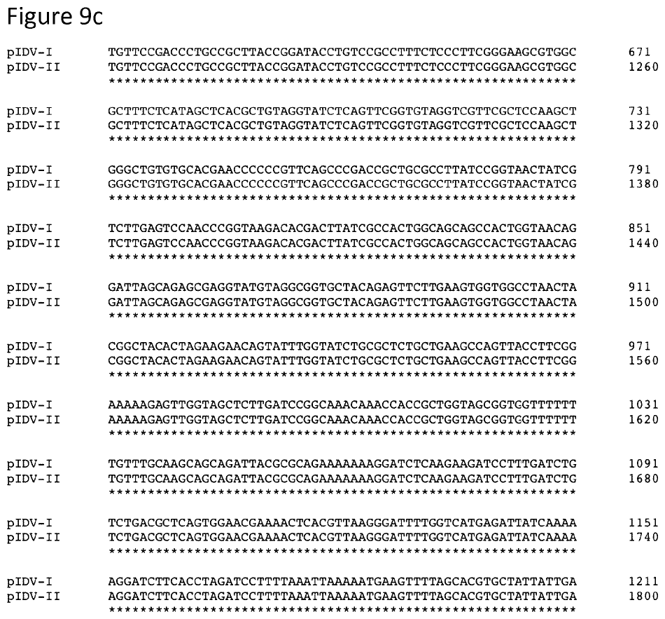

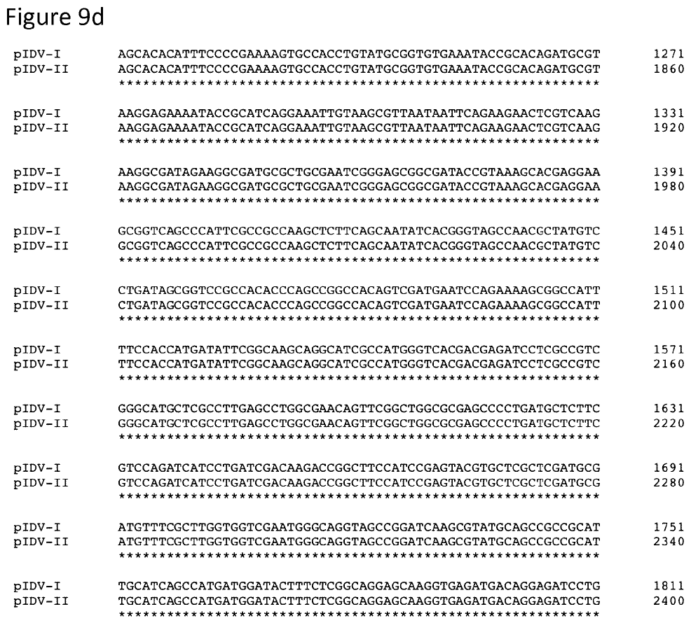

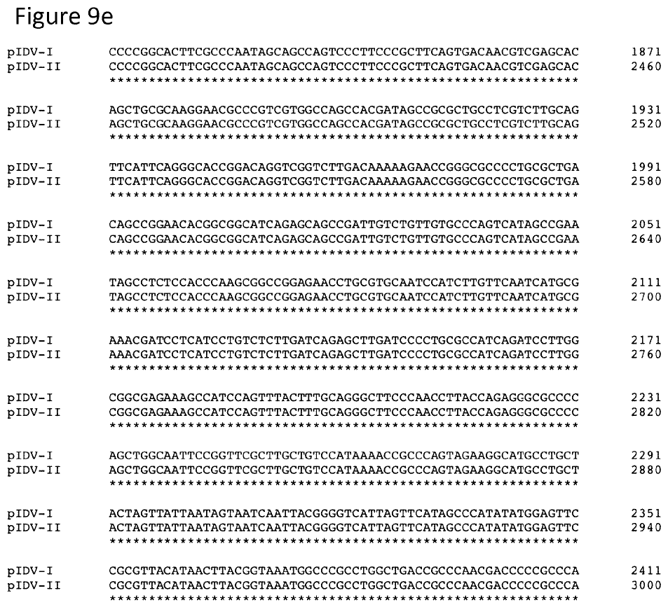

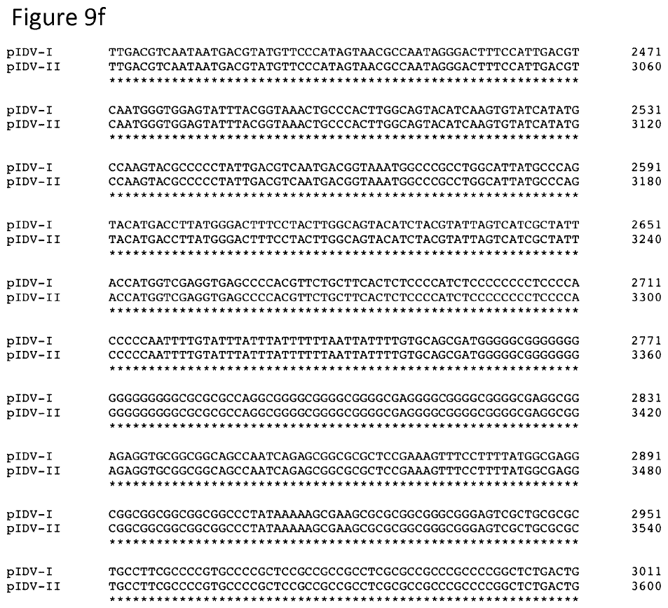

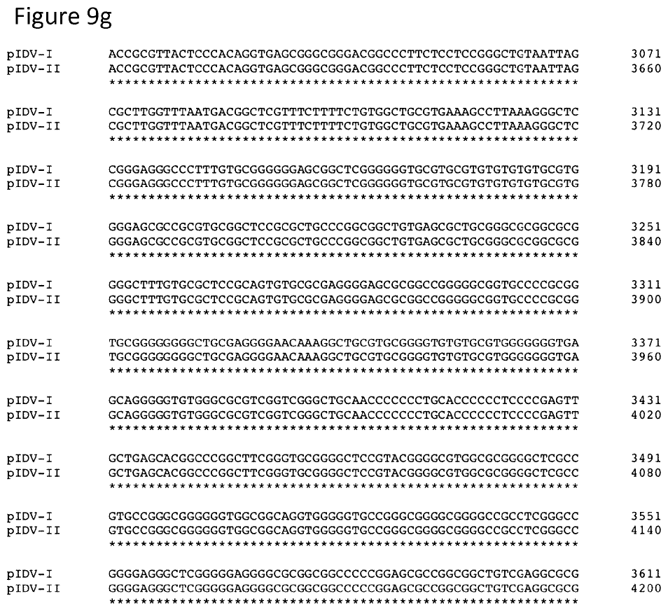

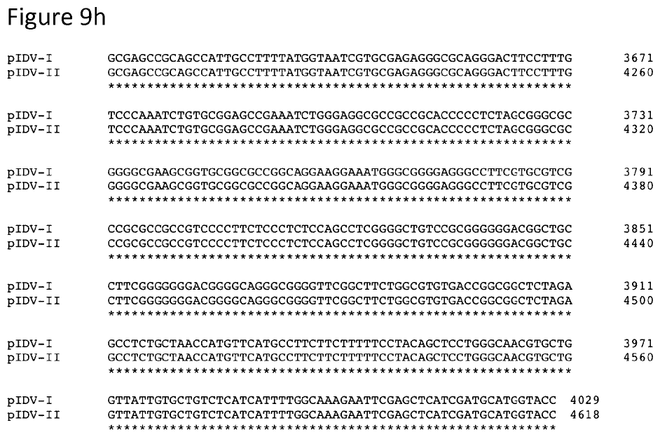

Our first attempt to remove the “Amp promoter” resulted in decreased expression from the plasmid. As such the Amp promoter was kept in the pIDV plasmid identified by SEQ ID NO:1. Subsequent attempts were proven successful with the generation of pIDV-I (SEQ ID NO:23) and pIDV-II plasmids (SEQ ID NO:24) (FIGS. 9a-9h).

### Reversion of ORI-Neo/Kan Cassette

In order to increase expression of antigens inserted into pIDV, the orientation of the ORI-Neo/Kan cassette was reversed. To accomplish this, we designed primers using SnepGene® software based on a reverse complement algorithm with a minimum of 15 matching base pairs (SEQ ID NO.:11 and SEQ ID NO.:12). The ORI-Neo/Kan cassette was then amplified and the pIDV plasmid was linearized at the Asel and HindIII sites. The amplified fragment and the cut plasmid were purified by Takara NucleoSpin™ PCR Clean-Up and Gel Extraction Kit, according to the manufacturer's instructions. Purified DNA was assembled using the NEB Gibson Assembly™ method based on manufacturer's guidelines and recommendations. Briefly, 100 ng of purified vector DNA was mixed with 3-fold excess of the ORI-Neo/Kan insert and was added to 10 μl of 2× Gibson Assembly Master Mix. To achieve a final reaction volume of 20 μl, the appropriate volume of water was added to the assembly mix. The assembly reaction was performed in a thermocycler at 50° C. for 60 minutes.

Assembled products were diluted 4-fold with HO prior to transformation, i.e., 5 μl of assembled product was mixed with 15 μl of H₂O. Three microliters of the diluted assembled product was then introduced into competent cells.

### Cloning of Inserts

The cDNA sequence of the gene(s) of interest was cloned at the Kpnl-BglII cloning site.

WO 2019/218091 PCT/CA2019/050686

Chemically Competent Cells Transformation

A 30 μl of chemically competent cells from Clontech Laboratories, Inc. (Stellar™) were thawed on ice for approximately 5 minutes and 3μl of diluted assembled product was added to competent cells, gently mixed and incubated on ice for 30 minutes. Heat shock was performed at 42° C. for 45 seconds followed by incubation on ice for 2 minutes. An aliquot of 850 μ1 of room temperature SOC media was added and the tube was incubated at 37° C. for 60 minutes while shaking at 250 rpm. An antibiotic selection plate was warmed in advance to 37° C. After incubation, 100 μl of the cells were spread by sterile loop on the LB bacterial agar plate containing 50 mg/ml Neomycin/Kanamycin selection antibiotics. The plate was incubated overnight at 37° C.

### Screening of Single Clones for Absence of Mutations

Ten single colonies of transformed bacteria were picked and grown for 14-16 hours at 37° C. on 5 ml of LB medium supplemented with 50 mg/ml Neo/Kan antibiotics, shaking at 250 rpm. After the incubation period, transformants were harvested by centrifugation at 6000 g for 10 minutes. Plasmid DNA Mini prep purification was performed by QIAGEN Plasmid Mini Prep kit. The resulting DNA was quantified by NanoDrop™ 2000 (Thermo Scientific) prior to sequencing. The sequencing primers utilized were designed so as to have 20-25 nucleotide overlap with a melting temperature (Tm) equal to or greater than 56° C. (assuming A-T pair=2° C. and G-C pair=4° C.) and to have a GC content of approximately 50%.

In addition to sequencing, plasmids were checked for proper insertion through restriction enzyme digestion with HindIII and Spel, and then visualized on 1% by agarose gel electrophoresis.

Cell Culture and Transfection

Vero E6 cells were cultured in DMEM (Dulbecco's Modified Eagle Medium) (Sigma) supplemented with 10% FBS (Foetal bovine serum), 2 mM L-glutamine, 100 U penicillin and 0.1 mg/ml streptomycin (Sigma). Vero E6 cells in a 24-well plate were transfected in triplicate with pIDV-eGFP using Lipofectamine™ 2000 (Life Technologies), as directed by the manufacturer. As a positive control for eGFP expression, Vero E6 cells were transfected with either pCAGGS-eGFP or pVAX1-eGFP.

After an overnight incubation, transfected cells were washed twice with 1× sterile PBS, followed by staining with green fluorescent dye 780 in order to distinguish between live and dead cells. The cells were incubated for 30 minutes at room temperature and then fixed with 200 μl of CytoFix™ reagent (BD Biosciences) and incubated an additional 1 hour at 4° C. in light protective conditions.

The FACS Calibur™ and CellQuest™ Pro software (BD Biosciences, San Jose, Calif.) were used to measure and analyse the fluorescence intensity of transfected cells. Of the 25,000 events evaluated per sample, only those events with the forward-scatter and side-scatter properties of single Vero E6 cells were used in the measurement of GFP fluorescence. The threshold between fluorescence-positive and fluorescence-negative was set such that >99.5% of transfected Vero E6 cells were considered fluorescence-negative.

### Software and Statistical Analysis

The “fluorescent volume” represents a summation of eGFP fluorescence within the sub-population of cells that were eGFP-positive (GFP+), and this was calculated to be equal to the “fraction of eGFP+cells in the sample population” times the “average fluorescent intensity of these eGFP+cells”. The coefficient of variation within groups of replicates was calculated to be 100% times the standard deviation of measurements divided by the mean of the measurements based on triplicates.

### Results

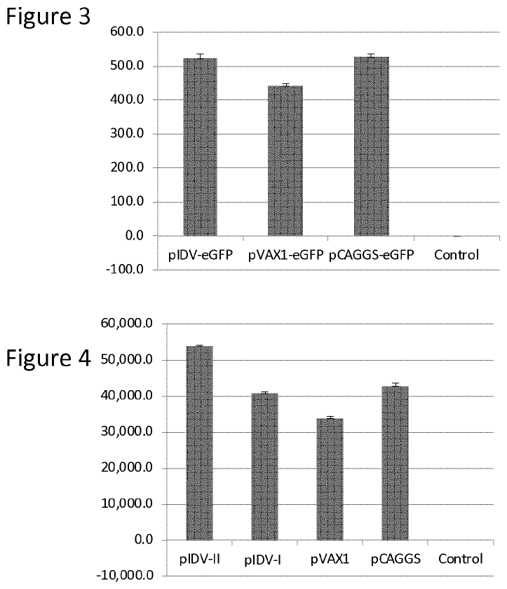

Using the methodology described above, Vero E6 cells were transfected with 2 μg of either pIDV-eGFP, pCAGGS-eGFP or pVAX1-eGFP using Lipofectamine™ 2000. Cells where harvested 24 hours post-transfection and eGFP expression was quantitated using fluorescence-activated cell sorting (FACs). Average and standard deviation of triplicate wells demonstrating eGFP expression in transfected cells is depicted in FIG. 3. We observed that pIDV-eGFP plasmid showed comparable eGFP expression as pCAGGS and higher eGFP expression in Vero E6 cells than pVAX1, the plasmid backbone most commonly used in clinical trials. Since pIDV comprises elements from the pVAX1™ vector, the pIDV plasmid is expected to be suitable for DNA vaccination.

### EXAMPLE 2—Construction of the pIDV-I and pIDV-II Vectors

### Materials and Methods:

The pIDV-II vector has been designed to allow easy insertion and subsequent high expression of exogenous genes in a wide variety of mammalian cells. The vectors share a common structure of a mammalian transcription unit composed of a promoter flanked 3′ by a polylinker, an intron, and a transcriptional termination signal which is linked to a pVAX1 backbone.

The pIDV-I plasmid was initially designed in silico based on insertion of 2919 bp fragment that includes CMV enhancer, cloning Chicken β-actin/Rabit β-globin hybrid promoter, site KpnI and BglII, β-globin polyadenylation signal and 3′ flanking region of rabbit β-Globin from recombinant plasmid pGAGGS at the sites of SpeI and HindIII, into pVAX1 plasmid which was in silico linearized with NruI and HIndIII restriction enzymes by Genius software. Thus, nucleotide 32-1054 which contains the CMV promoter, the T7 promoter, the multiple cloning sites and the bGH PA terminator were removed from pVAX1. Circularized plasmid was synthesized (GenScript).

Reversion of ORI-Neo/Kan Cassette and Deletion of AmpR promoter

In order to increase expression from the pIDV-I vector, the ORI-Neo/Kan cassette was reversed. To that effect, primers with at least 15 base pairs match were designed by SnepGene® software based on reverse complement algorithm. The ORI-Neo/Kan cassette was then amplified, and the pIDV-I plasmid was linearized at the Asel and HindIII sites. Amplified fragment and the cut plasmid were purified by Takara Nucleospin PCR Clean-Up and Gel Extraction Kit according to the manufacturer's instructions. Purified DNA was assembled by NEB Gibson Assembly method based on manufacturer's instructions.

As for the best cloning efficiency the purified DNA was optimized to −100 ng of vector with 3-fold of excess ORI-Neo/Kan insert and was added in to 10 of 2× Gibson Assembly mix, filed up with H₂O up to 20 μl of total reaction master mix. Reaction was performed in a thermocycler at 50° C. for 60 minutes.

Assembled products were diluted 4-fold with H₂O prior transformation, i.e. 5 μl of assembled products was mixed with 15 μl of H₂O. 3 μl of diluted assembled product was then introduced into competent cells.

In order to delete the AmpR promoter (76 bp) derived from pVAX1 vector along with the Ori-Neo/Kan cassette between the positions 1215-1290 bp, the two separate PCR reaction was performed where the Ori and Neo/Kan fragments were amplified separately. DNA was purified and NEB Gibson Assembly was performed based on manufacturer's instructions as described in above.

### Insertion of WPRE Fragment

To improve expression, the Woodchuck Hepatitis Virus Posttranscriptional Regulatory Element (WPRE) was inserted at position 7 to 595bp of pIDV-I thereby generating pIDV-II. This DNA sequence stabilizes post-transcriptional mRNA and thus increases expression as illustrated in FIG. 4 (compare pIDV-I and pIDV-II).

### Cell Culture and Transfection

VeroE6 cells were cultured in DMEM-Dulbecco's Modified Eagle Medium (Sigma) supplemented with 10% FBS -foetal bovine serum, 2 mM L-glutamine, 100 U penicillin and 0.1 mg/ml streptomycin (Sigma). VeroE6 cells were transfected in triplicates in 24 well plates using Lipofectamine 2000 (Life Technologies) as per manufacturer's instructions with empty plasmid (control), pIDV-I-eGFP, pIDV-II-eGFP, pVAX1-eGFP and pGAGGS-eGFP.

After overnight incubation, transfected cells were washed twice with 1× sterile PBS, followed by staining with green fluorescent dye 780 incubated for 30 minutes at room temperature. After incubation, cells were fixed with 200 μl of CytoFix reagent (BD Biosciences) and incubated an additional hour at +4° C. in light protective conditions.

A Becton Dickinson FACS Calibur and CellQuest Pro software (BD Biosciences, San Jose, Calif.) were used to measure fluorescence intensity of transfected cells. Of the 25,000 events evaluated per sample, only cells with the forward-scatter and side-scatter properties of single VeroE6 cells were used in measurements of GFP fluorescence. The threshold between fluorescence-positive and fluorescence-negative was set such that >99.5% of uninoculated VeroE6 cells were considered fluorescence-negative.

### EXAMPLE 3

The pIDV, pIDV-I and pIDV-II vectors are used to generate DNA vector expressing antigens from the Crimean Congo Hemorrhagic Fever virus (CCHF). Exemplary genes encoding CCHF antigens are provided in SEQ ID NOs:13-16 and SEQ ID NO:27 and are individually cloned into the vectors. The CCHF virus glycoproteins of SEQ ID NO:19-20 are derived from the CCHFV strain “Turkey”.

Experiments are performed to evaluate the cellular and humoral immune responses to the CCHF virus antigens in animals vaccinated with the DNA vectors.

The safety of the vaccine is determined by monitoring the systemic and local reaction to vaccination including site reactions and their resolution and clinical observation of the animals. Gross pathology will be performed at the end of the study.

The humoral response is determined using ELISA assay and the cellular response is determined by ELISPOT.

### Sample Size

For pre-clinical studies 8 groups of 10 female BALB/c mice aged between 6 to 8 weeks are used. Four (4) mice are tested for T-cell response and 6 for humoral immune response.

### Vaccination Dose and Prime Boost Schedule

In order to induce cellular and humoral immune response in mice, the DNA vaccines (pIDV-CCHF-GP-Tkk06-1, pIDV-CCHF-GP-Tkk06-2 (cocktail of pIDV-CCHF-Gn, pIDV-CCHF-Gc and pIDV-CCHF-NP); and empty backbone pIDV-Control) are administered by intramuscular injection.

Using this approach, the DNA vaccines are delivered to muscles by primary vaccination series followed by booster vaccination, i.e., entire dose of 200 μg is injected by two consecutive administrations into the exterior side of the mouse hind limbs. The volume and concentration of each injection is determined at 1 μg/ul or 100 μg/100 μl. The vaccine is administrated with 1 ml insulin syringes under isoflurane anesthesia, thus minimizing the puncture injury.

A baseline blood sample is collected from each mouse on Day −7 (in relation to the first dose of vaccine). Mice will subsequently be vaccinated on Days 0 and 28 (see schedule of events table). For testing the humoral immune response, mice are bled on Days 7, 14, 21, 27, 35, 49. Samples for humoral and cellular analysis are also obtained on Days 38 and 56 when mice are sacrificed. One seronegative animal serves as a control in each group in which the empty DNA vector is administrated without prime boosting.

TABLE 1
Schedule of Events

| | Day −7 | Day 0 | Day 7 | Day 14 | Day 21 | Day 27 | Day 28 | Day 35 | Day 38 | Day 49 | Day 56 |
|---|---|---|---|---|---|---|---|---|---|---|---|
| Vaccination | | X | | | | | X | | | | |
| Bleed | X | | X | X | X | X | | X | | X | |
| Sacrifice | | | | | | | | | X* | | X^{#} |

*Four mice from each group are sacrificed for cellular immune response analysis
^{#}All remaining mice are sacrificed for humoral immune response analysis at the end of the study

Four out of 10 mice are anesthetized and then euthanized 10 days after boost vaccination by cardiac puncture, and their spleen is removed to compare the T cell response against the CCHF antigens in the different groups.

The 6 remaining mice are euthanized by cardiac puncture followed by cervical dislocation 28 days after the boost vaccination (i.e., 56 days after first vaccination).

The serum samples obtained at the different intervals (−7, 7, 14, 21 & 27) are used to evaluate the production of antibodies against the CCHF GP and NP in the different groups.

The DNA vaccines are tested in farming animals according to a similar protocol.

### EXAMPLE 4

The pIDV, pIDV-I and pIDV-II vectors are used to generate DNA vectors expressing antigens from ticks. Exemplary transgenes are provided in SEQ ID NOs.:17-18 and 33. Exemplary antigens are provided in SEQ ID NO:34.

Experiments are performed to evaluate the cellular and humoral immune responses towards the tick antigens as outlined in Example 3.

### EXAMPLE 5

The pIDV-II plasmid was used to generate four individual vaccines expressing four different antigens.

The pIDV-II-CCHF-GP (SEQ ID NO:26) expresses the full length of whole CCHFV M segment ORF obtained from NCBI GenBank (Turkey isolate 812955; segment M, complete sequence GenBank Accession number KY362519.1). Prior to cloning into the pIDV-II vector the glycoprotein was human codon-optimized and fused to the signal sequence of Kozak followed by the first methionine of antigen at the 3′ amino-terminus situated after the plasmid promoter. To this end, the CCHF-GP from pUC57 vector (GeneScript) was amplified using a primer pair with at least of 19 bp homology to the pIDV-II plasmid. The insert was gel-eluted and further inserted into pIDV-II backbone cut by Kpn-BglII at position 4613-9688 by Gibson Assembly protocol (New England Biolabs NEB).

A plasmid containing the Ebola glycoprotein was also generated. pIDV-II -Ebola-GP-M06 (SEQ ID NO:29) expresses the full-length Ebola envelope glycoprotein (GP) which is available from NCBI GenBank (Zaire isolate).

Moreover, a pIDV-II plasmid encoding HIV envelope was also generated. To that effect, the envelope from the NL4.3 isolate was used as a proof of principle.

The resulting amplified insert, which contains gp120 and ectodomain of gp41 and a transmembrane protein, was cloned into the pIDV-II vector using Gibson Assembly cloning kit. In order to enhance initiation of translation, the Kozak sequence was included in primers so as to be located before the first methionine of the corresponding antigens.

For pIDV-II-HA86-p0 (SEQ ID NO:32) fused animal codon optimized HA86 antigen (Gene bank accession number: AF469170.1) derived from salivary gland of H. anatolicumanatolicum fused with 42 bp peptide sequence −p0 were cloned. This peptide originally derived from Rhipicephalus sanguineus acidic ribosomal protein P0 mRNA (GenBank accession number: KP087925.1). The HA86 protein represents an housekeeping gene, while the p0 peptide was found to be conserved only among of ectoparasites (including ticks, mosquitoes, Phebotomine sand flies etc.). In order to monitor protein expression, a His Tag was added at nucleotides 3388-3421 at the 3′ end of the protein.

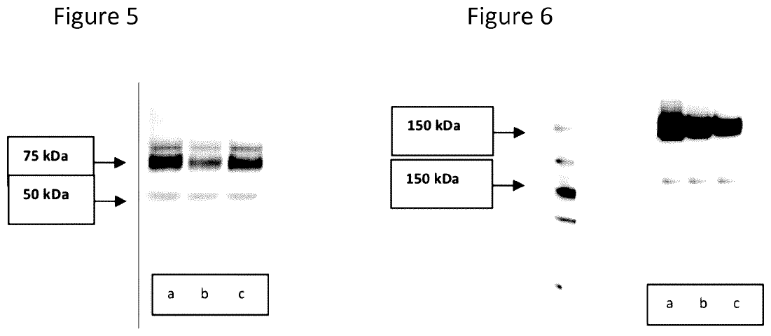

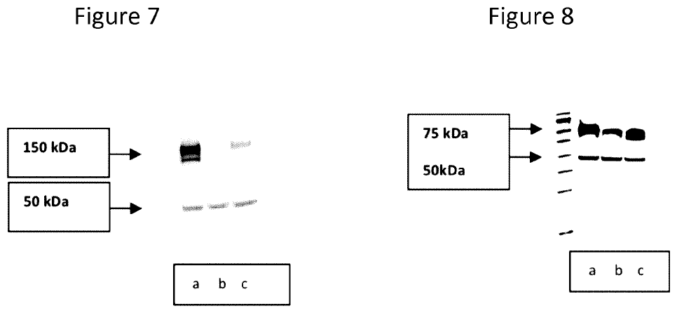

In order to compare the level of expression, all antigens were cloned in a similar fashion in two other plasmids: pVAX1 and pCAGGS as control groups. Antigen expression from the pIDV-II, pVAX1 and pCAGGS vectors was compared by Western Blot (FIGS. 5-8). The pIDV-II and pVAX1 vectors containing antigens (with the exception of tick antigen) were used in in vivo experiment.

### Chemically Competent Cells Transformation

A 30 μl of chemically competent cells (Clontech Laboratories, Inc.) were thawed on ice for about 5 minutes and 3 μl of diluted assembled product was added to competent cells, gently mixed and incubated on ice for 30 minutes. Heat shock was performed at 42° C. for 45 seconds followed incubation on ice for 2 minutes. A 850 μl of SOC media at room temperature was added and the tube was placed at 37° C. for 60 minutes of incubation at 250 rpm. Selection plate was warmed in advance to 37° C. After an incubation 100 μl of the cells were spread by sterile loop onto the into the LB bacterial agar plate containing 50 mg/ml Neo/Kanamicine selective marker. Plates were incubated for overnight at 37° C.

### Screening of Single Clones for Absence of Mutations

Ten single clones from transformed bacterial colonies were chosen and grown in shakers for 14-16 hours at +37 ° C., 250 rpm into 5 ml of LB medium supplemented with 50 mg/ml Neo/Kan antibiotics. After incubation, transformants were harvested by centrifugation at 6000 g for 10 minutes. Plasmid DNA Mini prep purification was performed by QIAGEN Plasmid Mini Prep kit. Nucleic acids were quantified by NanoDrop 2000 (Thermo Scientific) prior to sequencing. Enzymatic digestion with restriction enzymes and gel electrophoresis (1% by AGE) were used to confirm the identity of the vectors.

To exclude that no spontaneous mutations in the transgene has been introduced, selected clones were submitted for nucleotide sequencing.

Sequencing primers for all experiment were designed using a 19-25 nt overlap with a Tm equal to or greater than 56° C. (assuming A-T pair 32 2° C. and G-C pair=4° C.) and have a GC content of about 50%.

The concentration of oligonucleotides was adjusted at 1.6 04 and the concentration of plasmid at ≈50 ng/μl and submitted for Sanger sequencing. The plasmids having the best results of sequencing, especially for the absence of mutation, were selected for further evaluation of eGFP and for Western Blot respectively.

### Western Blot

At 24 h post-transfection, cell extracts were prepared in 50 mM Tris/HCl (pH 7.4), 5 mM EDTA, 1% Triton X-100 and Complete Protease Inhibitor cocktail. Cell lysates were centrifuged at 10 000 g for 10 min. The supernatant was quantified and 15 ug of each sample was mixed with sample buffer (10 M Tris/HCl (pH 6,8), 2% SDS, 10% glycerol, 5% β-mercaptoethanol, 0,005% bromophenol blue) and incubated at 56° C. for 10 min before electrophoresis in a Criterion Gel.

Western blot analysis was performed by using anti-CCHF mAb 11E7 (as primary antibodies for pre-GC-GCCCHF, 4F3 mouse anti-EBOV GPd™, mAb against Ebola (IBT Bioservices), for HIV mouse mAb against envelope glycoprotein 120 ID6 (AIDS reagent) and 1:2500 diluted His-Tag mAb-mouse (GenScript, Cat. No. A00186) for TickHA86 and incubated overnight at 4° C. with gentle agitation. As the loading control 1:20000 of secondary anti −a- Tubulin antibody (Sigma Aldrich) was used for each sample. Prior to adding the antibodies 3× washing steps were performed with 1XPBS-Tween 0.1% for 20, 5 and 5 minutes respectively. Goat anti-mouse human peroxidase-conjugated antibody was used followed by visualization with 4 ml total of substrate (Western blotting detection reagents Bio-Rad), while for HA86 containing backbone −Mouse IgG (H+L) Antibody, Human Serum Adsorbed and Peroxidase-Labeled antibody was used diluted at 1/20000. Results of protein expression are presented in FIGS. 5-8.

### Immunization of Mice

Groups of 7-10 mice aged 6-8 weeks (Charles River, Canada) were injected intramuscularly (IM) into the caudal thigh with 100 μg of pIDV-II and pVAX1 DNA vaccines containing the same antigen per animal diluted in Endotoxin-free TE buffer. Control animals received an equivalent volume of Endotoxin-free TE buffer. A total volume of 100 μl was introduced to each animal at two sites, each with 50 μl per limb. All mice were vaccinated with a single dose. Blood was obtained via subvein bleeds at day 0, 14 and 21 until the euthanasia (day 28). Serum was separated and kept frozen until analyzed. Three mice from each group were euthanized at day 10 for analysis of T-cell response.

### Mice Interferon-Gamma (IFN-γ) ELISpot Assay

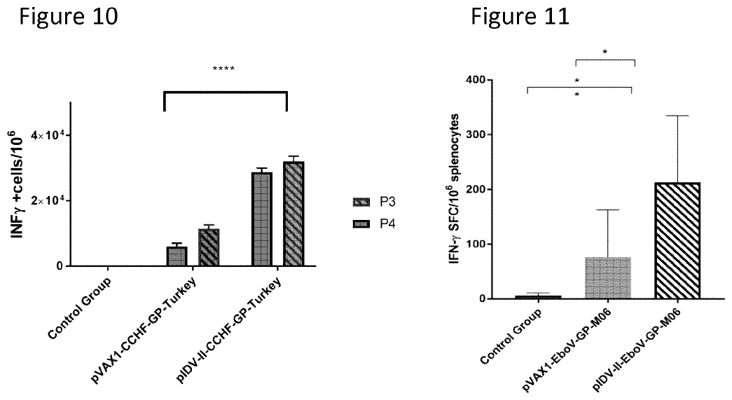

Splenocytes were assessed for CCHF and EboV antigen responses via IFN-γ enzyme-linked immunospot (ELISPOT) assay in accordance with manufacturer's instructions (BD Bioscience, San Jose, Calif.). Briefly, 96-well ELISPOT plates (Millipore, Billerica, Mass.) were coated overnight with anti-mouse interferon γ (IFN-γ) Ab, washed with phosphate-buffered saline, and blocked with 10% fetal bovine serum (FBS) in Roswell Park Memorial Institute medium (RPMI 1640). On day 10, splenocytes were harvested from 3 mice of each group of vaccinated mice to assess T-cell responses. A total of 5×10⁵ splenocytes in RPMI 10% FBS, 1% Pen/Strep and L-glutamine were plated per well and stimulated for 18-24 hours with 1 μg/mL of a peptide pools: for CCHF Partially overlapping peptide pools spanning the Gn and Gc of the CCHFV glycoprotein were applied in pools of 82 and 77 peptides designated as P3 and P4. For EboV the 176 peptides derived from a peptide scan through Envelope glycoprotein (GP/Mayinga-76) of Zaire Ebola virus (JPT, Innovative Peptide Solutions, Berlin, Germany) was used. 1% DMSO in RPMI and PMA 10 ng/ml/500 ng Ionomicynin RPMI was used as negative and positive controls respectively. Plates were placed for overnight incubation at 37° C. in a humidified incubator supplemented with 5% CO2. The following day, samples were extensively washed before incubation with biotinylated anti-mouse IFN-γ Ab. After incubation with streptavidin—horseradish peroxidase (HRP), IFN-γ-secreting cells were detected using AEC Chromogen (BD biosciences). Finally, spots were counted with an automated AID EliSpot Reader (FIGS. 10 and 11).

### ELISA CCHF

CCHF Viral like Particles (CCHF VLPs) were made as a reagent for ELISA. To that effect, production of IbAr 10200 strain of CCHF VLPs was performed based on improved protocol previously reported by Garrison et al (PLoS Negl Trop Dis, 11(9): e0005908, 2017).

Briefly, HEK 293T cells were propagated to 70±80% confluency in 10 cm² round tissue culture plates and then transfected with 10 μg pC-M Opt (IbAr 10200), 4 μg pC-N, 2 μg L-Opt, 4 μg T7-Opt, and 1 μg Nano-luciferase encoding minigenome plasmid using the Promega FuGENE HD transfection reagent according to manufacturer's instructions (Thermo Fisher Scientific). Three days post-transfection, supernatants were harvested, cleared of debris, and VLPs were pelleted through a cushion of 20% sucrose in virus resuspension buffer (VRB; 130 mM NaCl, 20 mM HEPES, pH 7.4) by centrifugation for 2 h at 106,750×g in an SW32 rotor at 4° C. VLPs were resuspended overnight in 1/200 volume VRB at 4° C., and then frozen at −80° C. in single-use aliquots. Individual lots of CCHF-VLP were standardized.

Mice sera were collected 28 days post-vaccination. Flat bottom ELISA plates were coated overnight at 4° C. with approximately 1 ng N equivalent of CCHF-VLP diluted in 1× PBS per 96-well plate. The following day, plates were washed and then blocked with 3% PBS/BSA 2 h at 37 ° C. All washes were done with 1× PBS containing 0.1% Tween-20. Plates were washed again, prior to being loaded with two different dilutions of mice sera in duplicate (dilution range 1:200 and 1:800). Serum dilutions were carried out in blocking buffer. Plates were incubated at 37° C. for 80 minutes prior to being washed again, and then incubated with a 1:4000 dilution of horse radish peroxidase (HRP) conjugated rabbit anti-mouse (Mandel) in PBST for 80 minutes at 37° C. Plates were washed again and then developed with TMB substrate (Sera-Care Inc.). Absorbance at 450 nm wavelength was measured with a microplate reader.

Individual naive sheep sera for each group collected from the same day point was used as an internal control on each assay group. A plate cut-off value was determined based on the average absorbance of the naive control starting dilution plus standard deviation. Only sample dilutions whose average was above this cut-off were registered as positive signal.

### ELISA EboV

Five mice per group were bled 1 day prior to immunization and every week after vaccination. Sera was kept frozen until analyzed. Corning Costarhalf area 96-well flat-bottom high-binding polystyrene microtiter plates were coated overnight at 4° C. with 30 μl/well of 2 μg/ml EBOV-VLP capture antigen (IBT Bioservices). Plates were blocked for 1 h with blocking buffer (KPL milk diluent/blocking, Sera care [150 μl/well] at 37° C.). Serum was serially diluted to 1:400 in KPL diluent buffer and 50 μl of the dilution was added to each well and incubated for 1 h at room temperature. The plates were washed six times with PBS-0.1%-Tween 20 (150 μl/well). 50 μl of a secondary antibody (goat anti-mouse IgG-HRP conjugate [1:2,000 dilution; Tonbo Bioscience]), was added to the wells and then incubated for 1 h at 37° C. The plates were washed 6 times with PBS-0.1%-Tween 20 (150 μl/well). Horseradish peroxidase substrate (KPL ABTS, Sera care) was then added (50 μl/well) and incubated at 37° C. for 30 min. Reaction was stopped with 50 μl/well of 1% SDS. The plates were read using a Biotek Synergy HTX microplate reader. The data are reported as the optical density at 405 nm (OD405).

### Software

Statistical significance of total IgG/avidity ELISA data was determined using two-way (Sidak's post hoc correction) ANOVA test for CCHF and one-way analysis of variance with Tukey's multiple comparison post-tests for EboV. Significance levels were set at a P value less than 0.05. All analyses were performed using GraphPad Prism software (La Jolla, USA), version 7.04.

### RESULTS

The data presented in FIG. 4 indicates that the pIDV-II plasmid showed higher eGFP expression in VeroE6 cell line in comparison with the other tested plasmids. In FIG. 4, the “fluorescent volume” represents a summation of eGFP fluorescence within the sub-population of cells that were eGFP-positive (GFP+), and this was calculated to be equal to the fraction of eGFP+ cells in the sample population times the average fluorescent intensity of these eGFP+ cells. The coefficient of variation within groups of replicates was calculated to be 100% times the standard deviation of measurements divided by the mean of the measurements based on triplicates.

### T-Cell Response in Vaccinated Mice

IFN-γ ELISpot responses from Balb/c mice immunized with pIDV-II-CCHF-GP-Turkey are compared to that of pVAX1-CCHF-GP-Turkey. Splenocytes from vaccinated mice were activated with peptide pools derived from GP of IbAr 10200 strain of CCHF peptide pool 3 (detecting G_{N}) and peptide pool 4 (detecting G_{C}). Patterned bars denote the number of spots against the peptide pool 3 while open bars shows spot number against peptide pool 4 respectively. As can be seen from FIG. 10, animals vaccinated with pIDV-II-CCHF-GP-Turkey shows higher T-cell response pattern compared to mice vaccinated with pVAX1 containing the same antigen. Results shown are the mean number of spot forming cells (SFC)±SD for 3 animals/group. Asterisks indicate statistically significant differences (****, p<0.005).

The Ebola glycoprotein (GP)-specific T-cell responses from vaccinated mice were assessed by the IFN-γ ELISpot. Splenic T-cells were stimulated with a pool of 176 peptides derived from a peptide scan through Envelope glycoprotein (GP/Mayinga-76) of Zaire Ebola virus and IFN-γ spot forming cells were enumerated after overnight incubation. As can be seen from FIG. 11, animals vaccinated with pIDV-II-EboV-GP-M06 developed stronger cellular immune response when compared to vaccinated animals from control pVAX1-EboV-GP-M06 groups. Results shown are the mean number of spot forming cells (SFC)±SD for 3 animals/group. Asterisks indicate statistically significant differences (**, p<0.005; *, p<0.05).

### Humoral Response at Day 0-28 Post Vaccination

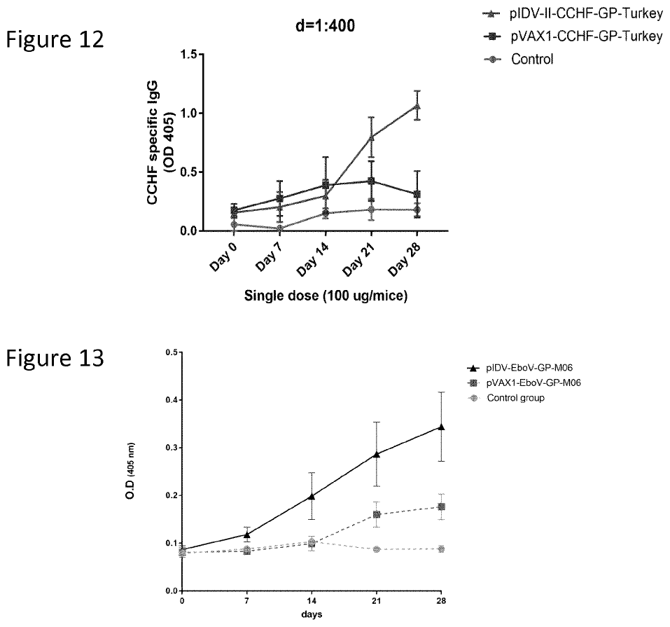

Results of FIG. 12 shows that only mice immunized with pIDV-II-CCHFV-GP developed IgG1 response with single dose. After single vaccination via IM route, CCHFV-specific antibodies were detected by ELISA against the CCHF-VLP only for mice vaccinated with pIDV-II-CCHF-GP-Turkey, while mice vaccinated with pVAX1-CCHF-GP-Turkey did not developed CCHF-specific antibodies. The CCHFV-specific IgG is shown in grouped mice following single vaccinations of 100μg/mouse. Collected sera at 7 days intervals from Balb/c mice vaccinated with only Endofree TE buffer (Control group) were tested concurrently and had no detectable signal. For mice immunized with pIDV-II-CCHF-GP-Turkey the highest serum titer was observed at day 28 after immunization. *Two-way ANOVA, confidence intervals were set to 95%., P-value=<0.0001.

Results of FIG. 13 shows that the titer of Ebola glycoprotein (GP)-specific IgG is higher after vaccination with pIDV-II-Ebov-GP-M06 compared to pVAX1-Ebov-GP-M06 by IM injection. Mice were immunized with 100 μg of the respective plasmids or Endofree TE buffer -control. The presence of Ebola GP-specific IgG in mouse sera was analyzed after vaccination by ELISA. Both CCHFV and EboV specific IgG ELISA titers were significantly increased at day 21 with high peak at day 28 after vaccination. However, it is possible that the maximum humoral response was not yet reached as the experiment was stopped at day 28.

The vectors disclosed herein and especially pIDV-II shows high gene expression patterns in both in vitro and in vivo experiments compared to pVAX1 vector which is the only platform licensed as DNA vaccine for human use.

The vectors disclosed herein were able to induce both cell-mediated and humoral immune responses for DNA encoding the CCHF and EboV antigens and assessed in mouse models, with fully functional innate immunity. The vectors are therefore useful to generate novel DNA vaccines with high gene expression in vitro and in vivo.

Advantageously the plasmids of the present disclosure are expected to meet the requirements of FDA for human use and shows high expression level in comparison to other DNA plasmids. Moreover, the plasmid of the present disclosure induce not only the humoral response but also cellular immune responses in Balb/c mice models with only single vaccine dose and only with entire ORF of CCHFV and EboV glycoproteins without any additional helper vaccines, which was used by other groups to express two proteins of distinct nature.

In summary, this study shows that the plasmids of the present disclosure, designed for DNA vaccination in human can trigger humoral and cellular immune responses.

SEQUENCE TABLE
A Sequence Listing in the form of a text file (entitled “16100-004-PCT_ST25_SequenceListing”,
created on May 18, 2019 of 142 kilobytes) is incorporated herein by reference in its entirety.

| SEQ ID NO: | Description | Comment |
|---|---|---|
| 1 | pIDV plasmid nucleotide sequence | BglII restriction site: nucleotides 1-6; KpnI |
| | | restriction site: nucleotides 4094-4099 |
| | | 57% GC |
| 2 | CMV enhancer-Position 2367-2732 | For SEQ ID NO: 2-9, nucleotide position is |
| | Total Length -366 bp | provided with reference to SEQ ID NO: 1 |
| 3 | Chicken β-actin promoter along with chimeric | |
| | intron-Position 2734-4023 Total Length -1290 bp | |
| 4 | β-globin poly(A) signal-Position 69-124 | |
| | Total Length -56 bp | |
| 5 | 3′ flanking region of rabbit β-Globin - | |
| | Position 125-450 Total Length -236 bp | |
| 6 | ORI -Position 485-1073 Total Length -589 bp | |
| 7 | AmpR promoter-Position 1215-1290 | Not present in pIDV-I and pIDV-II |
| | Total Length -76 bp | |
| 8 | NeoR/KanR-Position 1389-2183 | ″ |
| | Total Length -795 bp | |
| 9 | NeoR/KanR promoter-Position 2272-2321 | ″ |
| | Total Length -50 bp | |
| 10 | pVAX1 ™ plasmid sequence | |
| 11 | Neo/Kan Forward primer | |
| 12 | ORI Reverse primer | |
| 13 | Crimean Congo Hemorrhagic Fever Virus | |
| | glycoprotein precursor (CCHF GP-Turkey- | |
| | kk06) | |
| 14 | Ubiquitin- CCHF Glycoprotein GC | Ubiquitin sequence corresponds to |
| | | nucleotide 1-228 |
| 15 | Ubiquitin- CCHF Glycoprotein Gn | Ubiquitin sequence corresponds to |
| | | nucleotide 1-228 |
| 16 | CCHF Nucleoprotein (NP) | |
| 17 | Tick vaccine antigen #1 Rhipicephalus | |
| | appendiculatus salivary gland-associated | |
| | protein 64P mRNA, complete cds | |
| 18 | Tick vaccine antigen #2 Rhipicephalus | |
| | sanguineus acidic ribosomal protein P0 | |
| | mRNA, partial cds | |
| 19 | Crimean Congo Hemorrhagic Fever Virus | |
| | glycoprotein precursor (CCHF GP-Turkey- | |
| | kk06) amino acid sequence | |
| 20 | Ubiquitin- CCHF Glycoprotein GC amino | Ubiquitin sequence corresponds to |
| | acid sequence | amino acid 1-76 |
| 21 | Ubiquitin- CCHF Glycoprotein Gn amino | Ubiquitin sequence corresponds to |
| | acid sequence | amino acid 1-76 |
| 22 | CCHF Nucleoprotein (NP) -amino acid | |
| | sequence | |
| 23 | pIDV-I plasmid nucleotide sequence | |
| 24 | pIDV-II plasmid nucleotide sequence | WPRE position 7-595 |
| 25 | Woodchuck Hepatitis Virus | |
| | Posttranscriptional Regulatory Element | |
| | (WPRE) | |
| 26 | pIDV-II-CCHF-GP-Turkey nucleotide | CCFH Turkey antigen located at position |
| | sequence | 4613-9688 |
| 27 | CCHF GP-Turkey nucleotide sequence | |
| 28 | CCHF GP-Turkey amino acid sequence | Encoded by SEQ ID NO: 26 and 27 |
| 29 | pIDV-II-Ebola-GP-M06 nucleotide sequence | Kozak sequence, Ebola GP and M06 antigen |
| | | located at position 4613-6856 |
| 30 | Ebola-GP-M06 nucleotide sequence | |
| 31 | Ebola GP amino acid sequence | Encoded by SEQ ID NO: 29 and 30 |
| 32 | pIDV-II-HA86-p0 nucleotide sequence | HA86-p0 antigen and His tag located at |
| | | position 1370-3421 |
| 33 | HA86-p0 nucleotide sequence | Includes His tag |
| 34 | HA86-p0 amino acid sequence | Encoded by SEQ ID NO: 32 and 33 and |
| | | includes His tag |
| 35 | Probe binding sequence | |

SEQ ID NO: 36: Probe binding sequence wherein the probe binds to the nucleic acid sequence defined by N₁-TA-N₂ wherein N₁ is a nucleic acid sequence of 20 nucleotide or more that is complementary to a sequence at the 5′ end of the junction defined by nucleotides 2291 and 2292 of pIDV-I (SEQ ID NO: 23) and wherein N₂ is a nucleic acid sequence of 20 nucleotide or more that is complementary to a sequence at the 3′ end of the junction.

1-11. (canceled)

12. A vector having a nucleic acid sequence at least 80%, at least 85%, at least 90%, at least 95% or at least 99% identical to the nucleic acid sequence set forth in SEQ ID NO.:24.

13-14. (canceled)

15. The vector of claim 12, further comprising a gene encoding a protein or peptide.

16. The vector of claim 15, wherein the protein or peptide is an antigen.

17. The vector of claim 16, wherein the antigen is from a pathogen.

18. The vector of claim 16, wherein the antigen is a viral antigen, a bacterial antigen or a parasite antigen.

19. The vector of claim 18, wherein the viral antigen is from a human virus selected from the group of viruses from the Retroviridae family, Flaviviridae family, Togaviridae family, Picornaviridae family, Caliciviridae family, Astroviridae family, Coronaviridae family, Rhabdoviridae family, Filoviridae family, Paramixoviridae family, Orthomixoviridae family, Bunyaviridae family, Arenaviridae family, Reoviridae family, Papovaviridae family, Adenoviridae family, Parvoviridae family, Herpesviridae family, Poxviridae family, and Hepadnaviridae family.

20. The vector of claim 18, wherein the viral antigen is an antigen from HIV, from Ebola virus, from the Lassa virus, from the Nipah virus, from the Zika virus or from a coronavirus.

21. The vector of claim 18, wherein the parasite antigen is from a tick.

22. The vector of claim 16, wherein the antigen is a tumor specific antigen.

23. The vector of any one of claims 1 to 22claim 12, wherein the vector is circular or linear.

24. The vector of claim 15, wherein the vector also encodes an adjuvant molecule.

25. A composition comprising the vector of claim 15.

26. A pharmaceutical composition comprising the vector of claim 16, and a pharmaceutically acceptable carrier.

27. The pharmaceutical composition of claim 26, further comprising an adjuvant.

28. The pharmaceutical composition of claim 26, wherein the vector is formulated in nanoparticles.

29-30. (canceled)

31. A method of immunizing a host, the method comprising administering the pharmaceutical composition of claim 26 to the host.

32. The method of claim 31, wherein the host is a human or an animal.

33. (canceled)

34. The method of claim 31, wherein the pharmaceutical composition is administered by injection, by electroporation, intradermally, transdermally, intramuscularly or at a mucosal site.

35-54. (canceled)

55. A transgene comprising the sequence set forth in SEQ ID NO:17, SEQ ID NO:18, SEQ ID NO: 30, or SEQ ID NO: 33.

56-60. (canceled)

61. A vector expressing the transgene of claim 55, wherein the vector comprises the sequence set forth in SEQ ID NO:24 or a sequence at least 80%, at least 85%, at least 90%, at least 95% or at least 99% identical to SEQ ID NO:24.

62. A vaccine comprising one or more transgene of claim 55.

63. A vaccine comprising one or more vectors of 16.

64. A composition or pharmaceutical composition comprising the claim 63.

65. A method of immunizing a host comprising administering the pharmaceutical composition of claim 26 to the host.
